(19) United States
(12) Patent Application Publication
(10) Pub. No.: US 2004/0186052 A1
(43) Pub. Date: Sep. 23, 2004

(54) Cytomodulating peptides and methods for treating neurological disorders

(76) Inventors: Suhasini Iyer, San Ramon, CA (US); Roland Buelow, Palo Alto, CA (US); Mirella Emilova Lazarov, Palo Alto, CA (US); Timothy Fong, Moraga, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/693,331

(22) Filed: Oct. 24, 2003

Related U.S. Application Data
(60) Provisional application No. 60/421,297, filed on Oct. 24, 2002.
(60) Provisional application No. 60/431,420, filed on Dec. 5, 2002.
(60) Provisional application No. 60/470,839, filed on May 15, 2003.

(51) Int. Cl. A61K 38/17

(52) U.S. Cl. 514/12

(57) ABSTRACT

Compositions and methods are provided for inhibiting neuronal cell death and the loss of neuronal contacts resulting from acute and chronic neurological disorders, including neurodegenerative and neuroinflammatory diseases. The subject compositions and methods utilize RDP-58 compositions capable of providing a direct neuroprotective effect on neuronal cells in conjunction with the inhibition of autoimmune and inflammatory processes.

This application claims priority to provisional application serial no. 60/421,297, filed 24 Oct. 2002, and provisional application serial No. 60/431,420, filed 5 Dec. 2002, and provisional application serial No. 60/470,839, filed 15 May 2003, each of which is explicitly incorporated herein in its entirety by reference.

### FIELD

The present disclosure relates generally to compositions and methods for treating neurological disorders, and in particular to improved therapies for multiple sclerosis, HIV-associated dementia, Alzheimer's disease, Huntington's disease, Parkinson's disease, amyotrophic lateral sclerosis, stroke, epilepsy, retinal injury, auditory injury, peripheral neuropathy, injury from nervous system trauma, and a number of other neurological disorders characterized by one or more of neuronal injury, neuronal dysfunction, neuronal death, glial injury, glial dysfunction, and glial death.

### BACKGROUND

Neurological disorders, including chronic neurodegenerative diseases such as Alzheimer's disease and Parkinson's disease, and acute disorders such as stroke, affect a wide variety of cell populations in the nervous system and vary in time of onset, progression, and clinical presentation. Acute and chronic neurological disorders are associated with loss of sensory, motor, and cognitive abilities, as well as a high level of mortality. Despite their devastating effects, few effective treatments are available for the vast majority of these disorders, and most available treatments are directed to the management of symptoms, rather than the inhibition of disease.

While the causes of most neurological disorders are not known, neuronal death and the loss of neuronal contacts are pathological features common to many disorders. Moreover, studies have revealed that common biochemical mechanisms governing cell death operate in a variety of neurological disorders (Troy et al., J. Neurosci Res., 69:145-150, 2002; Yuan et al., Nature, 407:802-809, 2000; Friedlander et al., Cell Death Differ., 5:823-831, 1998; Thomas et al., Exp. Neurol., 133, 263-272, 1995; Troost et al., Neuropathol. Appl. Neurobiol., 21:498-504, 1995; Smale et al., Exp. Neurol., 133:225-230, 1995.) Thus, despite their varied etiologies, many neurological disorders appear to converge at biochemical pathways regulating cell death. Molecules functioning at such points of convergence and/or downstream in biochemical pathways are attractive therapeutic targets.

Studies have implicated mitogen activated protein kinases (MAPKs) in cell death in the nervous system. In particular, the MAPKs “JNK” and “p38” have been shown to play a role in neuronal death under a variety of conditions.

The JNK protein kinases are encoded by three genes, Jnk1, Jnk2, and Jnk3. Jnk1 and Jnk2 are expressed in a wide variety of tissues, while Jnk3 is selectively expressed in the brain (Dong et aL, Science, 270:1-4, 1998). JNK mediates many of its effects by phosphorylating a number of transcription factors, such as c-Jun, CREB, Elk-1, and ATF2.

A great deal of evidence supports the role of JNK in neuronal death and neurological disorders. Mice with targeted disruption of the JNK3 gene are protected from excitotoxin-induced neuronal apoptosis. In ischemic models, JNK activity is increased in cells impacted by infarct. JNK activity is also increased following kainic acid treatment, and an increase in JNK pathway activation is observed following electrical induction of seizures. JNK also appears to play a role in naturally occurring developmental cell death in a variety of neuronal populations, for example, in neurons of the substantia nigra. For review, see Harper and LoGrasso, Cellular Signaling, 13:299-310, 2001; Friedlander, N EngI J Med., 348:1365-1375, 2003.

The JNK pathway inhibitor “CEP-1347” has been shown to promote neuronal survival and reduce loss of function in a variety of conditions. For example, CEP-1347 reduces functional deficits and promotes the survival of ChAT neurons of the nucleus basalis following insult with ibotenic acid. The JNK-dependent apoptosis observed in this neuronal population suggests that JNK inhibitors may have utility as therapeutics for the treatment of Alzheimer's disease. In the MPTP model of Parkinson's disease, CEP-1347 promotes the survival of dopaminergic neurons in the substantia nigra. For review, see Harper and LoGrasso, Cellular Signaling, 13:299-310, 2001.; Friedlander, N EngI J Med., 348:1365-1375, 2003. CEP-1347 is currently in Phase II clinical trials for the treatment of Parkinson's disease. CEP-1347 has also been shown to reduce functional deficits and prevent hair cell and cochlear neuron death associated with trauma, suggesting uses for JNK inhibitors in the treatment of inner ear injuries (Pirvola et al., J. Neurosci., 20:43-50, 2000).

In addition, U.S. 6,288,089 discloses JNK inhibitors that reduce the death of mesencephalic neurons following serum withdrawal and following exposure to 6-hydroxy-dopamine (6-OHDA). Further disclosed is the ability of JNK inhibitors to reduce functional deficits following striatal lesion, and to enhance the survival of neurons transplanted into the striatum.

Additionally, in vitro experiments have shown the following: JNK is activated in PC12 cells and sympathetic neurons upon NGF withdrawal; constitutive activation of the JNK pathway in sympathetic neurons causes apoptosis in the presence of trophic factor; in primary striatal cultures, glutamate causes toxicity that is preceded by an increase in JNK activity. For review, see Harper and LoGrasso, Cellular Signaling, 13:299-310, 2001; Friedlander, N Engl J Med., 348:1365-1375,2003. Further, JNK activation is reportedly involved the AMPA-induced excitotoxic death of oligodendrocytes, implicating JNK in the death of glial cells as well as neurons (Liu et al., J. Neurochem., 82:398409, 2002).

Five isoforms of p38 have been identified, particularly p38α (Han et al, Science, 265:808-811, 1884; Lee et al, Nature, 372:739-746,1994), p38-β (Jiang et al., J. Biol. Chem., 271:17920-17926,1996), p38-γ (Li et al., Biochem. Biophys. Res. Commun., 228:334-340, 1996), p38-δ(Jiang, Y. et al., J. Biol. Chem. 272: 30122-30128,1997; Wang et al., J. Biol. Chem., 272:23668-23674, 1997) and p38-2 (Stein et al., J. Biol. Chem., 272:19509-19517, 1997). P38α and p38β are highly expressed in the brain. P38 modulates the activity of various transcription factors including ATF2, Elk-1, and MEF2C, and has been implicated in apoptosis (Thellung et al., Neurobiol. Dis., 9:69-81, 2002).

Du et al., (Proc. Natl. Acad. Sci., 98:14669-14674, 2001) have reported that the p38 inhibitor “minocycline” prevents nigrostriatal dopaminergic neuron death in the MPTP model of Parkinson's disease. Minocycline is believed to posses antiinflammatory activity as well as direct neuroprotective activity. Interestingly, minocycline is effective with oral administration despite its limited penetration of the blood-brain-barrier. It has also been reported that minocycline reduces infarct size and microglial activation following both focal and global ischemia (Yrjanheikki et al., Proc. Natl. Acad. Sci., 96:13496-13500, 1999; Yrjanheikki et al., Proc. Natl. Acad. Sci., 95:15769-15774, 1998). Moreover, minocycline exhibits neuroprotective activity in models of Huntington's disease (Chen et al., Nat. Med., 6:797-801, 2000), ALS (Zhu et al., Nature, 417:74-78, 2002) and nervous system trauma (Sanchez et al. Neurosurgery, 48:1393-1401, 2001). P38 has also been implicated in Alzheimer's disease. Recent analyses of postmortem brain tissues from individuals at different stages of disease progression have shown high levels of phosphorylated p38 in neuronal populations at early stages of the disease, particularly in the CA2 and CA1 regions of the hippocampus (Sun et al., Exp. Neurol., 183:394-405, 2003). These data suggest that p38 may be involved in the early stages of cellular degeneration in Alzheimer's disease.

Additional evidence supporting the involvement of p38 in neuronal apoptosis and neurological disorders includes the following: p38 and JNK exhibit cooperative activity in mediating the ceramide-induced death of primary cortical neurons (Willaime-Morawek et al., Neuroscience, 119:387-397, 2003); p38 activity increases following withdrawal of NGF from PC12 cells, and cell death is inhibited by the p38 inhibitor “PD169316”; glutamate-induced excitotoxicity is preceded by an increase in p38 activity in cerebellar granule neurons; the JNK/p38 inhibitors SB203580 and SB202190 both promote survival of a variety of neuronal subtypes deprived of trophic support. Additionally, the expression of phosphorylated p38 is increased in astrocyte-like cells in the core and penumbra regions of an infarct following focal ischemia, and in the hippocampus following global forebrain ischemia. Further, there is an increase in phosphorylated p38 in retinal ganglion cells undergoing cell death following axotomy of the optic nerve, and retinal ganglion cell death is reduced by the p38 inhibitor SB203580. P38 has also been implicated in ntiric oxide (NO) induced neuronal death, and SB203580 inhibits NO toxicity in primary neurons. For review, see Harper and LoGrasso, Cellular Signaling, 13:299-310, 2001; Friedlander, N EngI J Med., 348:1365-1375, 2003.

In addition to their involvement in neural cell death, JNK and p38 have been implicated in the regulation of immune responses and mechanisms of inflammation. Although the etiology of chronic neurodegenerative diseases such as multiple sclerosis and Guillain-Barre syndrome are unknown, it is clear that they involve inflammatory and autoimmune components. Inflammatory responses are also involved in acute neurological disorders such as stroke and brain trauma, and p38 has been shown to regulate the.expression of nitric oxide synthase, a molecule that plays a critical role in the excitotoxic death associated with stroke and traumatic injury. The immunosuppressive and anti-inflammatory agents presently used to treat these disorders have met with limited success. What is needed, therefore, are compositions and methods for ameliorating deleterious aspects of a neuroinflammatory responses and the cell dysfunction and death that follows.

It is an object of the present invention to provide JNK and p38 inhibitors capable of inhibiting the neuronal cell death and loss of neuronal contacts encountered in neurodegenerative diseases such as Parkinson's and Alzheimer's disease as well as other neurological disorders. It is a further object of the present invention to provide JNK and p38 inhibitors that serve a dual role in the treatment of chronic neuroinflammatory diseases such as MS and Guillain-Barre syndrome, and acute neuroinflammatory disorders such as stroke and nervous system trauma, by inhibiting autoimmune and inflammatory process while providing a direct neuroprotective effect.

### 3. ADDITIONAL RELEVANT LITERATURE

Buelow et al. Transplantation 59: 649-654 (1995) and references cited therein. Manolios et al., Nature Medicine 3: 84-88 (1997) describes oligopeptides derived by rational design. WO 95/13288 by Clayberger et al. describes HLA peptides capable of modulating T cell activity. References describing methods for designing compounds by computer using structure activity relationships include Grassy et al., J. of Molecular Graphics 13:356-367 (1995); Haiech et al. J. of Molecular Graphics 13:4648 (1995); Yasri et al. Protein Engineering 11: 959-976 (1996); Ashton et al. Drug Discovery Today 1:71-78 (1996); and lyer et al. Curr. Pharm. Des. 8: 2217-2229 (2002).

### 4. SUMMARY

Disclosed herein are RDP-58 compositions capable of modulating the biochemical activities of a variety of molecules involved in the transduction of a variety of biochemical signals in neural and other mammalian cell types. Also disclosed herein are methods using such RDP-58 compositions to modulate biochemical signals (signal transduction) and the cellular and physiological processes impacted thereby.

The RDP-58 compositions disclosed herein include compositions that are capable of modulating JNK activity, preferably kinase activity as directed at a substrate of JNK. The RDP-58 compositions disclosed herein also include compositions that are capable of modulating p38 activity, preferably kinase activity as directed at a substrate of p38. P38 and p38MAPK and grammatical equivalents are used interchangeably throughout the present disclosure. The RDP-58 compositions disclosed herein also include compositions that are capable of modulating TRAF activity, preferably binding activity as directed at a binding partner of TRAF, and/or kinase activity as directed at a substrate of TRAF. The RDP-58 compositions disclosed herein also include compositions that are capable of modulating IRAK activity, preferably binding activity as directed at a binding partner of IRAK, and/or kinase activity as directed at a substrate of IRAK. The RDP-58 compositions disclosed herein also include compositions that are capable of modulating AP-1 activity, preferably DNA-binding activity as directed at an AP-1 binding site in DNA, and/or transcription regulating activity as directed at an AP-1 responsive gene. The RDP-58 compositions disclosed herein also include compositions that are capable of modulating p53 activity, preferably DNA-binding activity as directed at a p53 binding site in DNA, and/or transcription regulating activity as directed at a p53-responsive gene. The RDP-58 compositions disclosed herein also include compositions that are capable of modulating NF-kB activity, preferably DNA-binding activity as directed at an NF-kB binding site in DNA, and/or transcription regulating activity as directed at an NF-kB responsive gene, and/or the nuclear localization of NF-kB. In a preferred embodiment provided herein are RDP-58 compositions having a combination of two or more such activities.

In one aspect, compositions and methods for inhibiting neural cell death are provided, wherein neural cells are contacted with a neuroprotective amount of an RDP-58 composition or mixtures thereof. The neural cells may be neuronal cells or glial cells, and the contacting may occur in vivo, in vitro and/or ex vivo.

In a further aspect, compositions and methods are provided for reducing neural cell death in a patient suffering from a neurological disorder, comprising administering to said patient a neuroprotective amount of an RDP58 composition. In one embodiment, the cell is a neuron. In another embodiment, the cell is a glial cell. In a third embodiment, the method is for reducing neuronal and glial cell death.

In an additional preferred embodiment provided herein is a method for reducing neural cell apoptosis. In one embodiment, the cell is a neuron. In another embodiment, the cell is a glial cell. In a third embodiment, the method is for reducing neuronal and glial cell apoptosis.

In another aspect, the invention provides methods for treating acute and chronic neurological disorders, comprising administering to a patient in need of such treatment an RDP-58 composition or mixtures thereof.

In one embodiment, the neurological disorder is an acute disorder.

In a preferred embodiment, the acute disorder involves inflammation.

In a preferred embodiment, the acute disorder involving inflammation is due to nervous system trauma, such as traumatic spinal cord injury or brain injury.

In another preferred embodiment, the acute disorder is ischemic or hemorrhagic stroke.

In another embodiment, the neurological disorder is a chronic disorder.

In a preferred embodiment, the chronic disorder involves inflammation.

In a preferred embodiment, the chronic disorder involving inflammation is a neuromuscular disorder.

In a preferred embodiment, the neuromuscular disorder is myasthenia gravis.

In a preferred embodiment, the chronic disorder involving inflammation is HIV-associated dementia.

In a preferred embodiment, the chronic disorder involving inflammation is a demyelinating disease.

In a preferred embodiment, the demyelinating disease is selected from the group consisting of multiple sclerosis, acute disseminated encephalomyelitis, optic neuromyelitis, transverse myelopathy, chronic inflammatory demyelinating polyneuropathy (CIDP), and Guillain-Barre syndrome.

In another embodiment, the chronic disorder involving inflammation is chronic fatigue syndrome.

In a preferred embodiment, the chronic disorder is a neurodegenerative disease selected from the group consisting of Parkinson's disease, Alzheimer's disease, amyotrophic lateral sclerosis (ALS; Lou Gehrig's disease) and Huntington's disease.

In a preferred embodiment provided herein are methods to treat neurological disorders, which involve administering to a patient an RDP-58 composition or mixtures thereof, wherein administration is by intracerebral injection.

In another preferred embodiment herein, administration involves permeabilization of the blood brain barrier.

In another preferred embodiment herein, the method of administering an RDP-58 composition or mixtures thereof may vary, and at least one RDP-58 composition used comprises a targeting moiety that provides for the localization of RDP-58 at its target tissue.

In another aspect, compositions and methods of treatment are provided involving the use of a second agent in combination with an RDP-58 composition or mixtures thereof. More than one agent may be used in combination with an RDP-58 composition (i.e., third agent, fourth agent, etc.). Preferred second agents include growth factors and cells, including oligodendrocytes and precursors thereof, glial cells that support oligondendrocytes, a variety of neuronal subtypes including dopaminergic neurons and cholinergic neurons, and a variety of neuronal precursor cells including neural stem cells and fetal brain cells. Preferred among growth factors are members of the fibroblast growth factor family (e.g., basis and acidic FGF), platelet derived growth factor (PDGF), glial cell line-derived neurotrophic factor (GDNF) and insulin like growth factors (IGF-1 and IGF-2). For an example of the use of IGF to treat ALS, see Kaspar et al., Science, 301:839-842, 2003. Other preferred second agents include IL-4, IL-6, IL-10, IL-13, transforming growth factor-β (TGF-β ), neurotrophin-3 (NT-3), ciliary neurotrophic factor (CNTF), leukemia inhibitory factor (LIF), brain derived neurotrophic factor (BDNF), nerve growth factor (NGF), and granulocyte colony stimulating factor (G-CSF). These factors may be used individually or in various combinations with RDP-58 compositions to treat a neurological disorder.

Also disclosed herein are methods of treatment involving the use of an RDP-58 composition with one or more other compounds used for demyelinating diseases, including immunosuppressants (e.g., mitroxanthone, cyclosphosphamide, methotrexate, azathioprine, cyclosporin, FK-506, etc.), immunomodulators (e.g., interferons β-1α and β-1α, and glatiramer acetate, etc.) and corticosteroids (e.g., prednisone, methyl prednisolone, dexamethasone, etc.).

Also disclosed herein are methods of treatment involving the use of an RDP-58 composition with one or more other JNK and/or p38 inhibitors. Preferred inhibitors include minocycline, VX-608, SB203580, CEP-1347, SB-202190 and PD169316.

Various pharmaceutical compositions are provided. The pharmaceutical compositions each comprise one or more RDP-58 compositions and a pharmaceutically acceptable carrier. Included in a preferred embodiment are pharmaceutical compositions that comprise a moiety that enhances transport of an RDP-58 composition across the blood brain barrier, the moiety being part of the carrier or conjugated to an RDP-58 composition. Also included in a preferred embodiment are pharmaceutical compositions comprising an RDP-58 composition in combination with one or more of the afore-mentioned second agents and/or one or more other compounds used for demyelinating diseases.

In a preferred embodiment provided herein are methods for the preparation of a medicament for use in the treatment of an acute or chronic neurological disorder.

In one embodiment, the neurological disorder is an acute disorder.

In a preferred embodiment, the acute disorder involves inflammation.

In a preferred embodiment, the acute disorder involving inflammation is due to nervous system trauma, such as traumatic spinal cord injury or brain injury.

In another preferred embodiment, the acute disorder is ischemic or hemorrhagic stroke.

In another embodiment, the neurological disorder is a chronic disorder.

In a preferred embodiment, the chronic disorder involves inflammation.

In a preferred embodiment, the chronic disorder involving inflammation is a neuromuscular disorder.

In a preferred embodiment, the neuromuscular disorder is myasthenia gravis.

In a preferred embodiment, the chronic disorder involving inflammation is a demyelinating disease.

In a preferred embodiment, the demyelinating disease is selected from the group consisting of multiple sclerosis, acute disseminated encephalomyelitis, optic neuromyelitis, transverse myelopathy, chronic inflammatory demyelinating polyneuropathy (CIDP), and Guillain-Barre syndrome.

In another embodiment, the chronic disorder involving inflammation is chronic fatigue syndrome.

In a preferred embodiment, the chronic disorder is a neurodegenerative disease selected from the group consisting of Parkinson's disease, Alzheimer's disease, amyotrophic lateral sclerosis (ALS; Lou Gehrig's disease) and Huntington's disease.

Also provided herein are compositions and methods for increasing the survival of a neuron, or a precursor thereof, that is transplanted into the nervous system of a recipient. In one embodiment of the subject method, the neuron or precursor thereof is contacted with an RDP-58 composition prior to transplant. This contact may be in vitro, in vivo in the donor, or ex vivo, or a combination thereof. In another method, the neuron or precursor thereof is contacted with an RDP-58 composition following transplant. In another embodiment of the method, the neuron or precursor thereof is contacted with an RDP-58 composition both before and following transplant.

In the methods of treatment provided herein, the administration of the RDP-58 composition may be by any convenient means, including by direct application or administration of the composition or, where applicable, a nucleic acid encoding the desired RDP-58 composition or the RDP-58 peptide component thereof to the afflicted cell population or tissue or organ. Alternatively, the RDP-58 compositions may be administered indirectly via routes which result in delivery of the composition to the afflicted tissue or organ.

### 5. BRIEF DESCRIPTION OF DRAWINGS

FIG. 1: Effect of treatment with RDP58 at different administration times on the development of EAE. Female Lewis rats were immunized with intradermal injections of inoculum (equal volumes of 2 mg myelin basic protein/ml PBS with complete Freund's adjuvant) into each footpad. At various time points after immunization (day 1, 4, 7, & 10), animals received icv administration of RDP58 (150 μg) in a 5% Mannitol/sterile water solution. Animals were scored daily for clinical symptoms on a 0-4 predetermined scale. Mean clinical scores were determined from each treatment group (n=5).

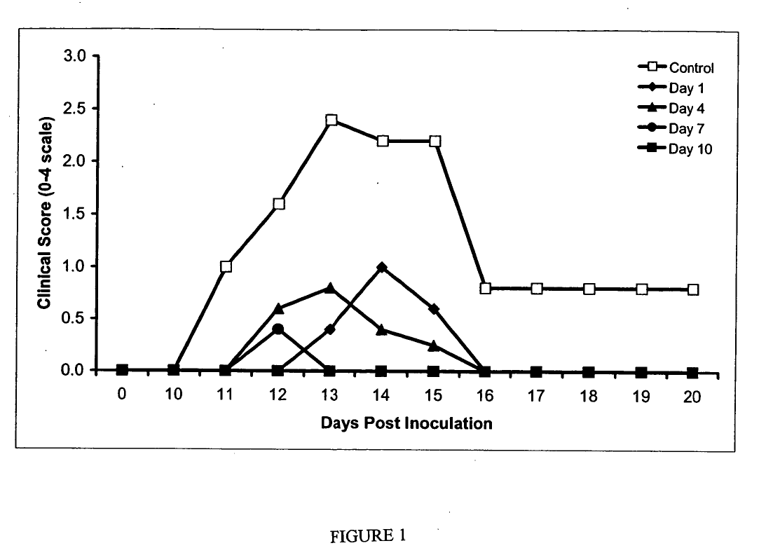

FIG. 2: Dose-dependent effects of RDP58 treatment on EAE-associated disease activity and weight loss. Female Lewis rats were immunized with intradermal footpad injections of inoculum containing 2 mg myelin basic protein with complete Freund's adjuvant. Ten days after immunization, animals received various doses of RDP58 (50, 15, or 5 mg) by icv injection. A) Animals were scored daily for clinical symptoms on a 04 predetermined scale. Mean clinical scores were determined from each treatment group (n=9). B) RDP58 reduces the change in body weight associated with EAE induction. The data is graphed as % body weight to illustrate changes compared to the value for each animal at the experimental starting point. Error bars in both graphs represent the standard error of the mean at each time point.

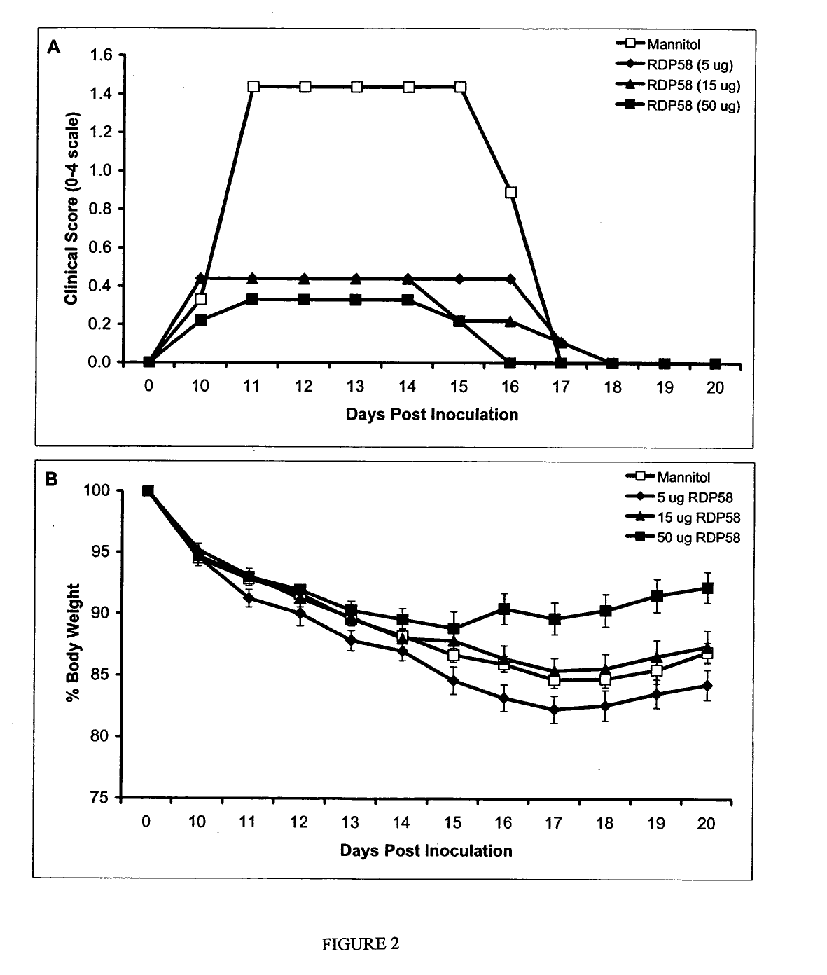

FIG. 3: Effect of RDP58 treatment on inflammatory infiltration in the spinal cord. Spinal cords from control (A) and RDP58-treated (B) animals were fixed thirteen days after immunization and examined for lymphocyte infiltration by H&E staining. Representative slices were chosen from the analysis of two different animals in each group. Arrows indicate sites of concentrated infiltration.

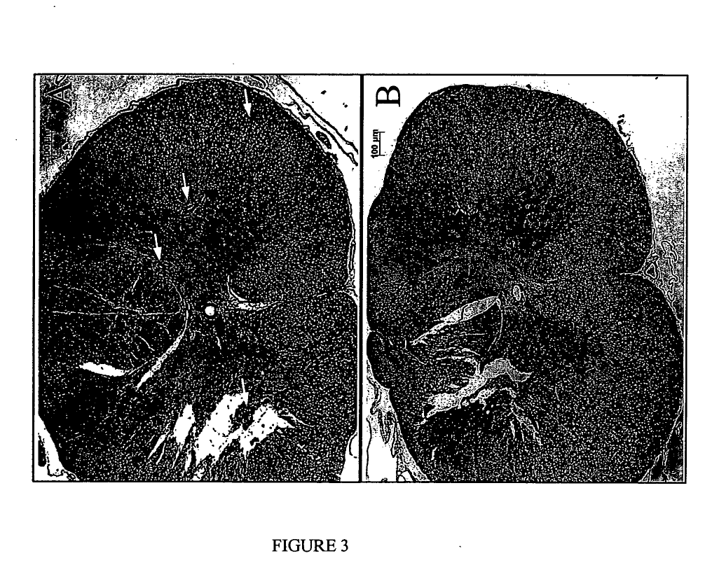

FIG. 4: Clinical scores of RDP58 and control treatment groups used in cytokine analysis. Female Lewis rats were immunized with intradermal footpad injections of an inoculum containing equal volumes of MBP+CFA. Eight days after immunization, animals received 50 mg of RDP58 by icv injection. Animals were scored daily for clinical symptoms on a 0-4 predetermined scale. Mean clinical scores were determined from each treatment group (n=12).

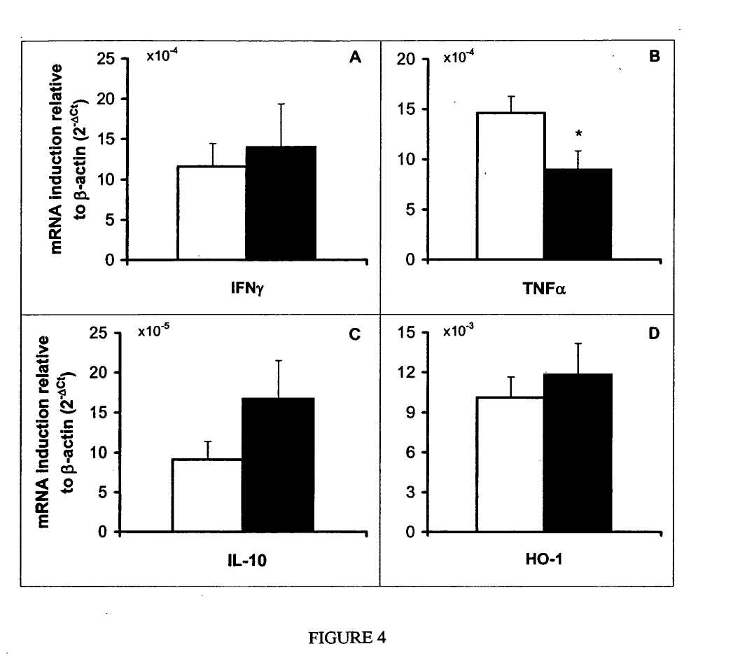

FIG. 5: TNFα and other cytokine mRNA levels altered by RDP58 treatment. Brain RNA from RDP58-treated and control animals was analyzed for changes in gene expression thirteen days after immunization. Gene expression levels represented in absolute terms of 2^{-ΔCt} to illustrate differences between RDP58- and Mannitol-treated animals (n=9, mean+S.E.M.). TNFα significantly reduced in response to RDP58 treatment (P<0.05).

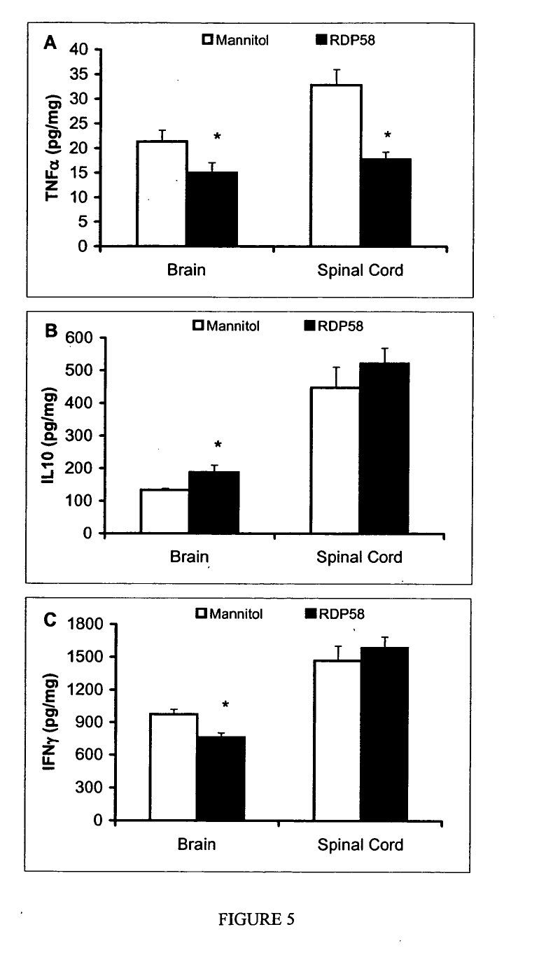

FIG. 6: Effects of RDP58 treatment on cytokine protein levels. Cytokine levels in spinal cord (A) and brain (B) from RDP58-treated and control animals (n=12 per treatment group) were analyzed by ELISA thirteen days after immunization. Protein levels expressed as mean pg/mg with error bars indicating S.E.M. C) IFNγ levels in brain and spinal cord as determined by ELISA. D) TNFα was measured using a bioassay with L929 cells. * denotes significant difference between treatment groups (p<0.05).

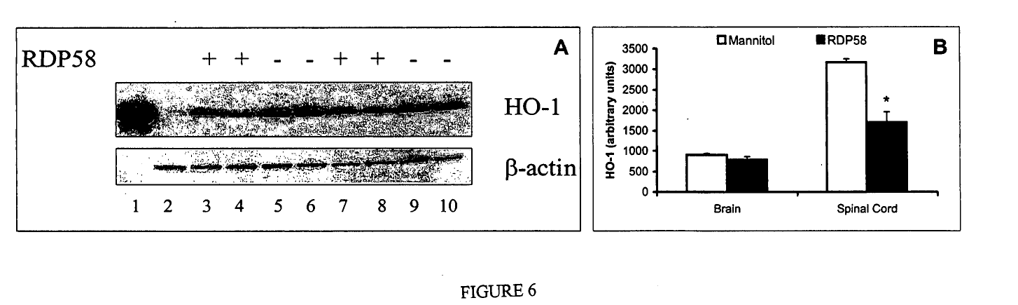

FIG. 7 shows Table I, summarizing results establishing that intracerebroventricular administration of RDP58 has beneficial effects on acute EAE, reducing several parameters of the disease: incidence, mean clinical scores, disease index, and severity.

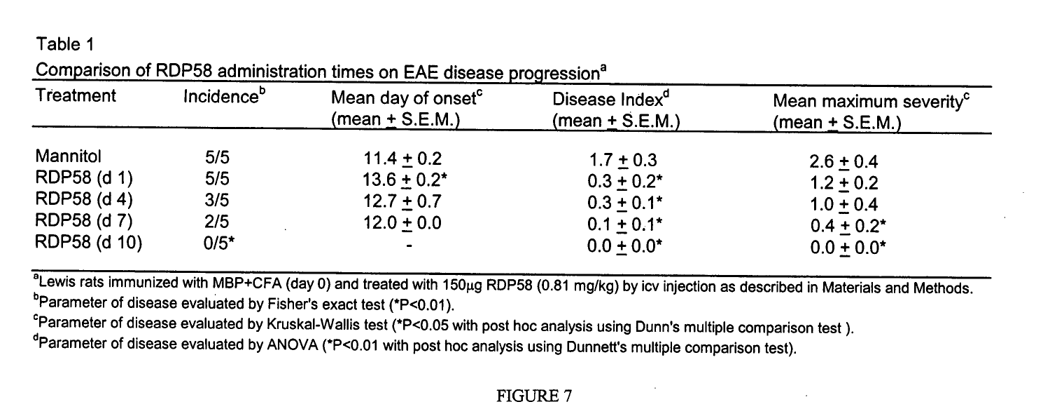

### 6. DESCRIPTION OF PREFERRED EMBODIMENTS

It has now been found that RDP-58 compositions are capable of modulating, particularly inhibiting, the activity of at least MEK, MEKK, JNK, p38MAPK (alternatively referred to as p38), NF-kB and AP-1 in mammalian cells, including mammalian neural cells. These molecules have been implicated in neurological disorders and/or neural cell death. The RDP-58 compositions disclosed herein find use in the inhibition of their activity, and the treatment of a variety of neurological disorders including autoimmune and inflammatory neurological disorders.

Among the neurological disorders treated by the methods disclosed herein are acute neurological disorders including stroke (ischemic and hemorrhagic) and nervous system trauma. The inhibition of p38 and NF-kB is desirable for limiting infarct size, cell death, cytokine production, and neurological deficits following from stroke.

Also among the neurological disorders treated by the methods disclosed herein are the chronic neurodegenerative diseases Parkinson's disease, Alzheimer's disease, Huntington's disease, and amyotrophic lateral sclerosis. The inhibition of p38 and JNK is desirable for the treatment of these neurodegenerative diseases.

Also among the neurological disorders treated by the methods disclosed herein are autoimmune and neuroinflammatory neurological disorders, including MS and Guillain-Barre syndrome. The presently disclosed RDP-58 compositions have the advantageous characteristic of being JNK and p38 inhibitors that possess anti-inflammatory activity, and serve a dual role in the treatment of chronic neuroinflammatory diseases such as MS and Guillain-Barre syndrome, and acute neuroinflammatory disorders such as stroke and nervous system trauma, by inhibiting autoimmune and inflammatory process while providing a direct neuroprotective effect.

Multiple sclerosis is generally considered an autoimmune and inflammatory disease of the central nervous system that selectively targets oligodendrocytes. The disease appears typically in early adulthood and shows variable prognosis. In relapsing-remitting MS (RRMS), which affects about 85% of MS patients, manifest symptoms last for several days, then stabilize and improve over the course of several days or weeks. The symptoms include sensory dysfunction, optic neuritis, limb weakness, gait ataxia, brain-stem symptoms, and bowel dysfunction. Patients also display increased fatigue, termed Uhthoff symptom, which correlates with increases in body temperature. The disease is characterized pathologically by the presence of focal plaques in the brain's white matter, representing regions of demyelination. In RRMS, remissions occur for varying lengths of time between relapses.

In the primary progressive form of MS (PPMS), the clinical course of the disease begins with chronic progressive myelopathy (i.e., an upper motoneuron syndrome of the legs). Symptoms typically worsen and evolve into quadriparesis, cognitive decline, visual loss, and brain-stem syndromes.

The majority of patients with the relapsing-remitting form eventually develop symptoms similar to the primary progressive form, indicating progression into what is classified as secondary progressive MS (SPMS). This progressive loss of neurological function appears to arise from irreversible injury to axons, glial scarring, and the death of oligodendrocytes.

Treatments for MS have included the use of immunosuppressants, corticosteroids, and immunomodulators. Immunosuppressants, such as cyclosporin, suppresses the immune system as a whole, but have undesirable side effects and have not demonstrated clear efficacy in the treatment of MS. Cytotoxic immunosuppressive agents, such as mitoxanthrone and cyclophosphamide, are highly cytotoxic and have systemic side effects. Corticosteroids generally suppress immune system activation and reduce levels of pro-inflammatory cytokines. These drugs are non-specific immunosuppressants that are used to treat acute active phases of multiple sclerosis, but they show limited effectiveness in the treatment this chronic condition. Immunomodulators include interferon β-1a (Avonex® and Rebif®), interferon β-1b (Betaseron® and Betaferon®), and glatiramer acetate (Copaxone®)). The interferons appear to suppress T-cells and inflammatory cytokines. Glatiramer acetate, a mixture of synthetic peptides, is hypothesized to mimic myelin basic protein and appears to induce specific suppressor T-cells and suppress effector T-cells. Trial studies with these immunomodulators on the relapsing-remitting form of MS show reductions in disease episodes in patients treated with interferon β-1b and glatiramer acetate but not with interferon β-1a (Khan, O. A. et al. Mult. Scler. 7: 349-53 (2001)).

Several lines of evidence suggest that the cytokine “TNFα” is involved in the etiology of multiple sclerosis. TNFα levels are elevated in serum and cerebrospinal fluid from MS patients, and TNFα is cytotoxic to oligodendrocytes in vitro. In transgenic animals that overexpress TNFα in the CNS, demyelinating disease characterized by oligodendrocyte apoptosis, demyelination, and infiltration of CNS by lymphocytes and macrophages is observed.

Therapies for MS have also been directed towards reducing the levels of TNFα. Pirfenidone, a non-peptide agent that reduces TNFα synthesis and blocks TNFα receptors, has been shown to reduce symptoms and slow the progression of MS (Walker, J. E. Mult. Scler. 7: 305-12 (2001)). However, other evidence suggests that treatments directed to the inhibition of TNFα have an insignificant effect on the progression of MS or may worsen the symptoms. For instance, anti-TNFα antibody therapy in secondary progressive MS was ineffective in reducing demyelinating plaques (Skurkovich, S. Mult. Scler. 7: 277-84 (2001)). Treatments with TNF antagonists (Lenercept® or Ethanercept®) had no effect on the disease and resulted in the exacerbation of symptoms (Neurology 53: 457-65, 1999). Similarly, use of anti-TNF monoclonal antibody—Infliximab®—for rheumatoid arthritis is correlated with incidences of demyelinating disease symptoms in some patients (Robinson, W. H. et al. Arthritis Rheum. 44: 1977-83 (2001)); Mohan, N. Arthritis Rheum. 44: 2862-69 (2001); and Sicotte, N. L. et al. Neurology 57: 1885-88 (2001)). In addition, mice lacking a functional TNF gene develop demyelination and CNS inflammation upon induction of EAE (Liu, J. Nat. Med. 4: 78-83 (1998)). Treatment with TNFα reduces the severity of the disease in these mice. Thus, according to the prior art, the use of anti-TNF agents may be ineffectual or possibly even contra-indicated. Further, despite the availability of several immunomodulatory and immunosuppressive drugs on the market, these have only demonstrated moderate effectiveness and efficient treatments for MS patients are still in need. Thus, therapies that promote cell survival in addition to providing anti-inflammatory activity are highly desirable.

Experimental autoimmune encephalomyelitis (EAE) is an inflammatory disease of the central nervous system, which can be induced in several species by either active immunization with myelin components or passive transfer of activated, myelin-specific CD4+ T cells. EAE has been used as a model of MS in an attempt to elucidate the mechanisms of MS and test potential therapeutic agents. The complex pathogenesis of MS includes inflammation, demyelination, and potentially disabling focal lesions leading to destructive pathological changes in the central nervous system (CNS). Most patients suffering from MS have an initial relapsing-remitting course of disease for several years before it takes on a progressive course of irreversible neurologic disability. Many of these clinical features can also be recapitulated in the various animal species used in EAE studies but on a much shorter time scale. It has been documented that relapses in MS patients correlate with inflammation and demyelination, whereas restoration of nerve function and remission are usually accompanied by resolution of inflammation and remyelination. However, the initial mechanism for the onset of disease is largely unknown.

Parkinson's disease is a chronic neurodegenerative disease characterized by the progressive loss of tyrosine hydroxylase(TH)-expressing dopaminergic neurons of the substantia nigra. Symptoms of Parkinson's disease include tremor, rigidity and bradykinesia. In advanced stages, patients exhibit problems with speech and a decline in cognitive function. A number of cellular and growth factor therapies have been tested with varied results. For example, see Dauer et al., Neuron, 39:889-909, 2003. Direct infusion of GDNF into the putamen was recently reported to improve patients' performance (Gill et al., Nat. Med., 9:589-595, 2003). In addition, the JNK inhibitor “CEP-1347” is currently in Phase II clinical trials for the treatment of Parkinson's disease.

Amyotrophic lateral sclerosis (ALS) is a chronic neurodegenerative disease characterized by the progressive loss of motor neurons of the brain, brain stem, and spinal cord. Clinical presentation begins typically in the fifth decade, and life expectancy following clinical presentation is typically four years. To date, the only treatment for ALS is riluzole, which extends survival by less than a year. The trophic factor “IGF” is a promising candidate therapeutic for the treatment of ALS, see Kaspar et al., Science, 301:839-842, 2003.

Huntington's disease is a chronic neurodegenerative disease characterized by neuronal death in the striatum and cortex. Huntington's disease is an autosomal dominant genetic disease in which mutant alleles encode polyglutamine repeats in the protein “huntingtin”. Clinical presentation begins during the fourth or fifth decade, and patients typically survive 15 to 20 years following onset. The disease is characterized by a movement disorder (chorea), cognitive dysfunction, and psychiatric symptoms. Huntington's disease is fatal and to date no treatment is available.

In a preferred embodiment provided herein are methods to treat neurological disorders, which involve administering to a patient an RDP-58 composition or mixtures thereof.

By “treatment” herein is meant therapeutic or prophylactic treatment, or a suppressive measure for the disease or undesirable condition. Treatment encompasses administration of the subject peptides in an appropriate form prior to the onset of disease symptoms, after clinical manifestation of the disease, or administration after appearance of the disease to reduce disease severity, halt disease progression, or eliminate the disease. Demyelinating disease as used herein includes, but is not limited to, forms such as multiple sclerosis, acute disseminated encephalomyelitis, optic neuromyelitis, transverse myelopathy, Guillain-Barre syndrome, chronic inflammatory demyelinating polyneuropathy (CIDP), and others known in the art.

For use as treatment or prophylaxis, the oligopeptides may be used alone or in combination with other therapeutic agents. In this context, the oligopeptides used are either a single oligopeptide sequence, or an admixture of different oligopeptide sequences of the present invention, or as an admixture that includes natural analogs of the peptides of the present invention. In another aspect, the peptides are used with other therapeutic or pharmaceutically active agents used to treat the particular condition or the disease. With reference to demyelinating diseases, agents that may be useful in combination with the peptides include corticosteroids (e.g., prednisone, methylprednisolone, dexamethasone, etc.), immunosuppressants (e.g, cyclosporin, FK-506, mitroxantone, cyclophosphamide, methotrexate or azatioprine, etc.); and immunomodulators (e.g., interferon, including interferon -b1a and interferon-b1b, and glatiramer acetate, etc.).

In another preferred embodiment, the peptides of the invention are used with growth factors that promote survival and/or growth of cells affected by the autoimmune and inflammatory reaction. In the case of demyelinating diseases, these will include factors affecting survival or growth of oligodendrocyte precursor cells (e.g., O2-A cells), oligodendrocytes, or neural cells that support oligodendrocytes. An advantage of using combinations of the peptide and growth factors is that the peptide may limit the inflammatory response while the factors promote survival and growth of damaged cells, or generation of new oligodendrocytes.

In a preferred embodiment, growth factors shown to affect oligodendrocyte survival and growth, including basic fibroblast growth factor (bFGF), platelet-derived growth factor (PDGF), and insulin-like growth factors (e.g., IGF-1 and IGF-2), are used. PDGF can cause proliferation of oligodendrocyte precursor cells (O2A cells), recruit O2A precursor cells, and act as a survival factor for oligodendrocytes. Basic fibroblast growth factor may limit programmed cell death of oligodendrocytes while IGF-1 and IGF-2 can promote hypermyelination and cell survival.

Within the scope of growth factors are various cytokines and trophic factors that ameliorate inflammation and tissue damage, in particular regards to oligodendrocytes affected in demyelinating diseases or cells damaged by an inflammatory component of the disorder (see Barres, B.A. et al. Development 118: 283-295 (1993), hereby incorporated by reference). These include, but is not limited to, anti-inflammatory cytokines, such as IL4, IL-6, IL-10, IL-13; transforming growth factor-b (TGF-b); neurotrophin-3 (NT-3); ciliary neurotrophic factor (CTNF); leukemia inhibitory factor (LIF); brain derived neurotrophic factor (BDNF); nerve growth factor (NGF); and granulocyte colony stimulating factor (G-CSF). For example, cytokines IL-6, CTNF, and LIF are a related family of cytokines which promotes oligodendrocyte survival, in particular CNTF and LIF.

In another aspect, various combinations of growth factors and cytokines are used with the peptides. These include combinations of related factors, such as CNTF, LIF, and IL-6, or combinations that provide an added or synergistic effect on oligodendrocyte survival and growth. In one preferred embodiment, combinations between different groups of factors are used. These include combinations between groups of factors: (1) IGF-1 and IGF-2, (2) CNTF, IL-6, and LIF, and (3) NT-3, BDNF, and NGF.

The growth factors and cytokines may be prepared in a pharmaceutically acceptable form for delivery to the afflicted tissues and administered according to the methods described herein. Growth factors and cytokines could be in the form of full length proteins or biologically active peptides prepared from recombinant or natural sources, as is known in the art. They may also be expressed in the subject animal or tissue by introducing a nucleic acid that is capable of expressing the subject protein, for example in the form of plasmids or retroviral constructs, as described above and is known in the art. Growth factors and cytokines may be co-administered with the peptides described herein, or administered pre or post-treatment with the peptide.

As disclosed herein, the subject compositions and methods may be used to inhibit neural cell death, and reduce neural cell death in patients suffering from neurological disorders. As used herein, the term neural cell includes both neuronal cells and glial cells.

Also provided herein are methods for increasing the survival of a neuron, or precursor thereof. By precursor thereof is meant a cell that has the capacity to become a neuron, or a cell that can give rise to a progeny cell that has the capacity to become a neuron. Included are neural stem cells. Also included are cells that may transdifferentiate to become or give rise to a neuron.

RDP-58 Compositions

RDP-58 compositions suitable for use in the methods disclosed herein will generally comprise at least one peptide, polypeptide or oligopeptide capable of providing a neuroprotective effect. Particularly preferred are peptides selected from the family of RDP-58 peptides described in PCT Publication WO 98/46633, which are characterized therein as being capable of inhibiting the cytotoxic activity of lymphocytic cells, inhibiting the production of inflammatory cytokines and inflammatory responses associated with those cytokines, inhibiting the activity of heme-containing enzymes and delaying the onset of autoimmune disease in a mammal at risk of developing such a disease. As disclosed herein, it has now been found that such peptides also have the ability to modulate a variety of biochemical pathways and affect the cellular and physiological processes impacted thereby.

Suitable peptides for use in the compositions and methods provided herein have a variety of characteristics, and may be identified in a number of ways.

In the preferred embodiment provided herein are RDP-58 peptides that are neuroprotective. By neuroprotective is meant capable of increasing neural cell survival under at least one condition that otherwise would lead to neural cell death, and/or capable of reducing neural loss of function under at least one condition that otherwise would lead to greater loss of function. A number of assays for examining the neuroprotective activity of potential kinase inhibitors are known. For example, US2002/0058245 discloses assays for examining the ability of potential JNK inhibitors to promote neuronal survival in the presence of physiologically relevant toxic stimuli, including Huntingtin protein, APP-C-100 protein, and glutamate. In addition, the removal of trophic support from primary neurons or neural cell lines (including PC12 cells) in culture is well known in the art as an assay for determining the neuroprotective effect of agents of interest. In vivo assays, for example the ability of a putative RDP-58 composition to protect striatal neurons from a chemical lesion are known in the art and may also be used. Any of the foregoing assays may likewise be used to gauge a candidate peptide's ability to reduce and inhibit neural cell death. Similarly, well-known assays such as TUNEL labeling may be used to measure prevention of neuronal apoptosis.

As disclosed herein, the subject RDP-58 compositions are capable of providing a direct neuroprotective effect contemporaneously with the inhibition of inflammatory and autoimmune processes. Inhibition of inflammatory or autoimmune responses includes reducing or eliminating one or more symptoms associated with an inflammatory or autoimmune response, including in particular the production of inflammatory cytokines such as TNF-α, IFN-γ, IL-2 and/or IL-12. Assays for determining cytokine production and immune cell activity are well known to the skilled artisan and need not be repeated here. See, e.g., PCT Publication WO 98/46633.

In the preferred embodiment, the subject RDP-58 peptides comprise one or more of the cytomodulating peptides disclosed in co-pending U.S. Patent Applications U.S.S.N 09/028,083 & U.S.S.N. 08/838,916 as well as corresponding International application WO 98/46633, the disclosures of which are expressly incorporated herein by reference. In an especially preferred embodiment, the RDP-58 peptide comprises the sequence Arg-nL-nL-nL-Arg-nL-nL-nL-Gly-Tyr, where nL is norleucine and all amino acids other than glycine are the D-stereoisomer.

In the preferred embodiment, the core sequence of the RDP-58 peptide desirably comprises two basic amino acids separated by from three to four hydrophobic amino acids, particularly three hydrophobic amino acids, and particularly where the N-terminus is a basic amino acid. More desirably, the C-terminal amino acid is an aromatic amino acid, particularly tyrosine. Of particular interest is where at least one of the oligopeptide core terminal amino acids is an oligopeptide terminal amino acid, which may be in the monomeric or oligomeric form of the compound.

More particularly, the preferred RDP-58 peptides for use in the compositions and methods of the present invention comprise oligopeptides having the sequence B-X-X-X-B-X-X-X-J-Tyr, where B is a basic amino acid, preferably Lys or Arg, particularly Arg on at least one position, preferably at both positions; J is Gly, B or an aliphatic hydrophobic amino acid of from 5 to 6 carbon atoms, particularly Gly or B; and X is an aliphatic or aromatic amino acid. In one embodiment, at least three X amino acid residues are the same non-polar aliphatic amino acid, preferably at least four are the same non-polar aliphatic amino acid, more preferably at least five are the same non-polar aliphatic amino acid, and most preferably, all are the same non-polar aliphatic amino acid. In a preferred embodiment, the non-polar aliphatic amino acids are of from 5 to 6 carbon atoms, particularly 6 carbon atoms, particularly the non-polar aliphatic amino acids Val, lie, Leu, and nL. Thus, in some embodiments, X is any amino acid other than a charged aliphatic amino acid, and preferably any amino acid other than a polar aliphatic amino acid.

Of the six amino acids indicated by X in the B-X-X-X-B-X-X-X-J-Tyr peptide sequence, preferably at least 3 are aliphatic amino acids of from 5 to 6 carbon atoms, more preferably at least 4 are aliphatic amino acids of from 5 to 6 carbon atoms, most preferably at least 5 are aliphatic amino acids of 5-6 carbon atoms, more particularly 6 carbon atoms. In a preferred embodiment, the aliphatic amino acids are non-polar aliphatic amino acids of from 5 to 6 carbon atoms, particularly Val, lie, Leu, and nL. The other amino acids may be other uncharged aliphatic amino acids, particularly non-polar aliphatic amino acids or aromatic amino acids.

Compositions of particular interest will include an RDP-58 peptide having the sequence:

Arg-U-X-X-Arg-X-X-X-J-Tyr

wherein all of the symbols have been defined previously except U, which comprises an uncharged aliphatic amino acid or aromatic amino acid, particularly a non-polar aliphatic amino acid or aromatic amino acid.

The amino acids may be naturally occurring amino acids or D-isomers thereof. The peptides may have one or more D-stereoisomer amino acids, up to all of the amino acids. Additionally, the peptides may comprise oligomers of the subject peptides, particularly dimers thereof, or comprise a cyclic peptide, that is a ring structure, as further described below.

For the purposes of this invention, the amino acids (for the most part natural amino acids or their D-stereoisomers) will be broken down into the following categories:

1. Aliphatic

(a) non-polar aliphatic:

Gly, Ala, Val, nL, lle, Leu

(b) polar aliphatic:

(1) uncharged:

Cys, Met, Ser, Thr, Asn, Gln

(2) charged:

Asp, Glu, Lys, Arg

2. Aromatic:

Phe, His, Trp, Tyr

wherein Pro may be included in the non-polar aliphatic amino acids, but will normally not be included. “nL” represents norleucine, where the non-polar aliphatic amino acids may be substituted with other isomers.

Exemplary RDP-58 peptides include the following:

bc #

| | | | | | | | | | | |
|---|---|---|---|---|---|---|---|---|---|---|
| 1 | Arg | Leu | Leu | Leu | Arg | Leu | Leu | Leu | Gly | Tyr |
| 2 | Arg | Val | Leu | Leu | Arg | Leu | Leu | Leu | Gly | Tyr |
| 3 | Arg | Ile | Leu | Leu | Arg | Leu | Leu | Leu | Gly | Tyr |
| 4 | Arg | Leu | Val | Leu | Arg | Leu | Leu | Leu | Gly | Tyr |
| 5 | Arg | Leu | Ile | Leu | Arg | Leu | Leu | Leu | Gly | Tyr |
| 6 | Arg | Leu | Leu | Val | Arg | Leu | Leu | Leu | Gly | Tyr |
| 7 | Arg | Leu | Leu | Ile | Arg | Leu | Leu | Leu | Gly | Tyr |
| 8 | Arg | Leu | Leu | Leu | Arg | Val | Leu | Leu | Gly | Tyr |
| 9 | Arg | Leu | Leu | Leu | Arg | Ile | Leu | Leu | Gly | Tyr |
| 10 | Arg | Leu | Leu | Leu | Arg | Leu | Val | Leu | Gly | Tyr |
| 11 | Arg | Leu | Leu | Leu | Arg | Leu | Ile | Leu | Gly | Tyr |
| 12 | Arg | Leu | Leu | Leu | Arg | Leu | Leu | Val | Gly | Tyr |
| 13 | Arg | Leu | Leu | Leu | Arg | Leu | Leu | Ile | Gly | Tyr |
| 14 | Arg | Trp | Leu | Leu | Arg | Leu | Leu | Leu | Gly | Tyr |
| 15 | Arg | Leu | Trp | Leu | Arg | Leu | Leu | Leu | Gly | Tyr |
| 16 | Arg | Leu | Leu | Trp | Arg | Leu | Leu | Leu | Gly | Tyr |
| 17 | Arg | Leu | Leu | Leu | Arg | Trp | Leu | Leu | Gly | Tyr |
| 18 | Arg | Leu | Leu | Leu | Arg | Leu | Trp | Leu | Gly | Tyr |
| 19 | Arg | Leu | Leu | Leu | Arg | Leu | Leu | Trp | Gly | Tyr |
| 20 | Arg | Tyr | Leu | Leu | Arg | Leu | Leu | Leu | Gly | Tyr |
| 21 | Arg | Leu | Tyr | Leu | Arg | Leu | Leu | Leu | Gly | Tyr |
| 22 | Arg | Leu | Leu | Tyr | Arg | Leu | Leu | Leu | Gly | Tyr |
| 23 | Arg | Leu | Leu | Leu | Arg | Tyr | Leu | Leu | Gly | Tyr |
| 24 | Arg | Leu | Leu | Leu | Arg | Leu | Tyr | Leu | Gly | Tyr |
| 25 | Arg | Leu | Leu | Leu | Arg | Leu | Leu | Tyr | Gly | Tyr |
| 1NI | Arg | nL | nL | nL | Arg | nL | nL | nL | Gly | Tyr |

nL = norleucine

Other exemplary RDP-58 peptides are disclosed in PCT application serial number PCT/US98/07231, filed 10 Apr. 1998, U.S. patent application Ser. No. 08/838,916, filed 11 Apr. 1997, and U.S. patent application Ser. No. 09/028,083 filed 23 Feb. 1998, each being expressly incorporated herein in its entirety by reference. Generally, the term “RDP-58 peptide” as used herein is meant to encompass all of the foregoing peptide compounds.

In further embodiments, other known peptides such as HLA peptides and TCR peptides may be alternatively or additionally used in the subject invention as components of the subject RDP-58 compositions. These include HLA-B α1-domain, particularly the amino acids from 75 to 84 and variations of this sequence where not more than 2 amino acids are replaced (see, e.g., WO 95/13288 and Buelow et al., expressly incorporated herein by reference). Also included are sequences based on the human TCR-α transmembrane region consisting of that sequence and sequences having not more than 2 mutations from that sequence (see Australian Application Nos. PN 0589 and PN 0590, filed Jan. 16, 1995, expressly incorporated herein by reference). These sequences include 2 basic amino acids, where the 2 basic amino acids are separated by 4 aliphatic hydrophobic amino acids, although the application indicates that from 3 to 5 hydrophobic amino acids may be present. By mutation is intended each substitution of one amino acid for another or an insertion or deletion, each being counted as one mutation. Generally, the term “peptide” as used herein is meant to encompass all of the foregoing peptide compounds, as well as analogs, derivatives, fusion proteins and the like.

The subject peptides may be modified in a variety of conventional ways well known to the skilled artisan. For example, one or both, usually one terminus of the peptide, may be substituted with a lipophilic group, usually aliphatic or aralkyl, of from 8 to 36, usually 8 to 24 carbon atoms and fewer than two heteroatoms in the aliphatic chain, the heteroatoms usually being oxygen, nitrogen and sulfur. As further described below, the chain may be saturated or unsaturated, desirably having not more than 3 sites, usually not more than 2 sites of aliphatic unsaturation. Conveniently, commercially available aliphatic fatty acids, alcohols and amines may be used, such as caprylic acid, capric acid, lauric acid, myristic acid and myristyl alcohol, palmitic acid, palmitoleic acid, stearic acid and stearyl amine, oleic acid, linoleic acid, docosahexaenoic acid, etc. (see U.S. Pat. No. 6,225,444, hereby incorporated by reference). Preferred are unbranched, naturally occurring fatty acids between 14-22 carbon atoms in length. Other lipohilic molecules include glyceryl lipids and sterols, such as cholesterol. The lipophilic groups may be reacted with the appropriate functional group on the oligopeptide in accordance with conventional methods, frequently during the synthesis on a support, depending on the site of attachment of the oligopeptide to the support. Lipid attachment is useful where oligopeptides may be introduced into the lumen of the liposome, along with other therapeutic agents (e.g., BMPs) for administering the peptides and agents into a host. Increasing lipophilicity is also known to increase transport of compounds across endothelial cells and therefore useful in promoting uptake of such compounds from the intestine or blood stream into surrounding tissues.

The terminal amino group or carboxyl group of the peptide may be modified by alkylation, amidation, or acylation to provide esters, amides or substituted amino groups, where the alkyl or acyl group may be of from about 1 to 30, usually 1 to 24, preferably either 1 to 3 or 8 to 24, particularly 12 to 18 carbon atoms. This is done using conventional chemical synthetic methods. The peptide or derivatives thereof may also be modified by acetylation or methylation to alter the chemical properties, for example lipophilicity. Methods for acylating, and specifically for acetylating the free amino group at the N-terminus are well known in the art. For the C-terminus, the carboxyl group may be modified by esterification with alcohols or amidated to form —CONH₂, CONHR, or CONR, wherein each R is a hybroxycarbyl (1-6 carbons). Methods of esterification and amidation are done using well known techniques. Other modifications include deamination of glutamyl and asparaginyl residues to the corresponding glutamyl and aspartyl residues, respectively; hydroxylation of proline and lysine; phosphorylation of hydroxyl groups of serine or threonine; and methylation of amino groups of lysine, arginine, and histidine side chains (see T. E. Creighton, Proteins: Structure and Molecular Properties, W. H. Freeman & Co. San Francisco, Calif., 1983).

In additional embodiments, either or both the N- and C-terminus of the peptide may be extended by not more than a total of about 100, usually not more than a total of about 30, more usually not more than about 20 amino acids, often not more than about 9 amino acids, where the amino acids will have fewer than 25%, more usually fewer than 20% polar amino acids, more particularly, fewer than 20% which are charged amino acids. Thus, extensions of the above sequences in either direction are mainly done with lipophilic, uncharged amino acids, particularly non-polar aliphatic amino acids and aromatic amino acids. The peptides may comprise L-amino acids, D-amino acids, or mixtures of D and L amino acids. Exceptions to the number of amino acid extensions are contemplated when the oligopeptides are expressed as fusion or chimeric proteins, as described below.

The peptides may also be in the form of oligomers, particularly dimers of the peptides, which may be head to head, tail to tail, or head to tail, there being not more than about 6 repeats of the peptide. The oligomer may contain one or more D-stereoisomer amino acids, up to all of the amino acids. The oligomers may or may not include linker sequences between the peptides. When linker sequences are used, suitable linkers include those comprising uncharged amino acids and (Gly)n, where n is 1-7, Gly-Ser (e.g., (GS)ₙ, (GSGGS)ₙ and (GGGS)ₙ, where n is at least 1), Gly-Ala, Ala-Ser, or other flexible linkers, as known in the art. Linkers of Gly or Gly-Ser may be used since these amino acids are relatively unstructured, which allows interaction of individual peptides with cellular target molecules and limits structural perturbations between peptides of the oligomer.

Peptides may also be in a structurally constrained form such as cyclic peptides of from about 9-50, usually 12 to 36 amino acids, where amino acids other than the specified amino acids may be present as a bridge. Thus, for example, addition of terminal cysteines allows formation of disulfide bridges to form a ring peptide. In some instances, one may use other than amino acids to cyclize the peptide. Bifunctional crosslinking agents are useful in linking two or more amino acids of the peptide. Other methods for ring formation are described in Chen et al., Proc. Natl. Acad. Sci. USA 89:5872-5876 (1992); Wu et al., Protein Engineering 6:471-478 (1993); Anwer, et al., Int J. Pep. Protein Res. 36:392-399 (1990); and Rivera-Baeza, et al. Neuropeptides 30: 327-333 (1996); all references incorporated by reference. Alternatively, structurally constrained peptides are made by addition of dimerization sequences to the N- and C-terminal ends of the peptide, where interaction between dimerization sequences lead to formation of a cyclic type structure (see WO/0166565, incorporated by reference). In other instances, the subject peptides are expressed as fusions to other proteins, which provide a scaffold for constrained display on a surface exposed structure, such as a loop of a coiled-coil orβ-turn structure.

Depending upon their intended use, particularly for administration to mammalian hosts, the subject peptides may also be modified by attachment to other compounds for the purposes of incorporation into carrier molecules, changing peptide bioavailability, extend or shorten half-life, control distribution to various tissues or the blood stream, diminish or enhance binding to blood components, and the like. The subject peptides may be bound to these other components by linkers which are cleavable or non-cleavable in the physiological environment such as blood, cerebrospinal fluid, digestive fluids, etc. The peptides may be joined at any point of the peptide where a functional group is present, such as hydroxyl, thiol, carboxyl, amino, or the like. Desirably, modification will be at either the N-terminus or the C-terminus. For these purposes, the subject peptides may be modified by covalently attaching polymers, such as polyethylene glycol, polypropylene glycol, carboxymethyl cellulose, dextran, polyvinyl alcohol, polyvinylpyrrolidine, polyproline, poly(divinyl-ether-co-maleic anhydride), poly(styrene-c-maleic anhydride), etc. Water soluble polymers, such a polyethylene glycol and polyvinylpyrrolidine are known to decrease clearance of attached compounds from the blood stream as compared to unmodified compounds. The modifications can also increase solubility in aqueous media and reduce aggregation of the peptides.

Peptide Conjugates and Fusion Proteins

In another aspect, the RDP-58 peptide or other useful peptide is preferably conjugated to one or more small molecules for detection and isolation of the peptide, and to target or transport the peptide into specific cells, tissues, and organs. Small molecule conjugates include haptens, which are substances that do not initiate an immune response when introduced by themselves into an animal. Generally, haptens are small molecules of molecular weight less than about 2 kD, and more preferably less that about 1 kD. Haptens include small organic molecules (e.g., p-nitrophenol, digoxin, heroin, cocaine, morphine, mescaline, lysergic acid, tetrahydrocannabinol, cannabinol, steroids, pentamidine, biotin, etc.). Binding to the hapten, for example for purposes of detection or purification, are done with hapten specific antibodies or specific binding partners, such as avidin which binds biotin.

Small molecules that target the conjugate to specific cells or tissues may also be used. It is known that presence of a biotin-avidin complex increases uptake of such modified peptides across endothelial cells. Linkage of peptides to carbohydrate moieties, for example to a β-glycoside through a serine residue on the peptide to form a β-O linked glycoside, enhances transport of the glycoside derivative via glucose transporters (Polt, R. et al. Proc. Natl. Acad. Sci. USA 91: 7144-7118 (1994); Oh et al. Drug Transport and targeting, In Membrane Transporters as Drug Targets, Amidon, G. L. and Sadee, W. eds., pg 59-88, Plenum Press, New York, 1999). Both of these types of modifications are encompassed within the scope of the present invention.

The peptides may have attached various label moieties such as radioactive labels and fluorescent labels for detection and tracing. Fluorescent labels include, but are not limited to, fluorescein, eosin, Alexa Fluor, Oregon Green, rhodamine Green, tetramethylrhodamine, rhodamine Red, Texas Red, coumarin and NBD fluorophores, the QSY 7, dabcyl and dabsyl chromophores, BIODIPY, Cy⁵, etc.

In one aspect, the peptides are joined to a wide variety of other peptides or proteins for a variety of purposes. The peptides may be linked to other peptides or proteins to provide convenient functionalities for bonding, such as amino groups for amide or substituted amine formation, e.g., reductive amination; thiol groups for thioether or disulfide formation; carboxyl groups for amide formation; and the like. Of particular interest are peptides of at least 2, more usually 3, and not more than about 60 lysine groups, particularly polylysines of from about 4 to 20, usually 6 to 18 lysine units, referred to as multiple antigenic peptide system (MAPS), where the subject peptides are bonded to the lysine amino groups, generally at least about 20%, more usually at least about 50%, of available amino groups, to provide a multipeptide product (Butz, S. et al. Pept. Res. 7: 20-23 (1994)). In this way, molecules having a plurality of the subject peptides are obtained where the orientation of the subject peptides is in the same direction; in effect one has a linking group to provide for tail to tail di- or oligomerization.

In another aspect, other naturally occurring or synthetic peptides and proteins may be used to provide a carrier immunogen for generating antibodies to the subject peptides, where the antibodies serve as reagents for detecting the peptides or for identifying other peptides having a comparable conformation. Suitable carriers for generating antibodies include, among others, hemocyanins (e.g., Keyhole Limpet hemocyanin-KLH); albumins (e.g., bovine serum albumin, ovalbumin, human serum albumin, etc.); immunoglobulins; thyroglobulins (e.g., bovine thyroglobulin); toxins (e.g., diptheria toxoid, tetanus toxoid); and polypeptides such as polylysine, as described above, or polyalanine-lysine. Although proteins are preferred carriers, other carriers, preferably high molecular weight compounds, may be used, including carbohydrates, polysaccharides, lipopolysaccharides, nucleic acids, and the like of sufficient size and immunogenicity. In addition, the resulting antibodies may be used to prepare anti-idiotypic antibodies which may compete with the subject peptides for binding to a target site. These anti-idiotypic antibodies are useful for identifying proteins to which the subject peptides bind.

In another aspect, the peptides are conjugated to other peptides or proteins for targeting the peptide to cells and tissues, or adding additional functionalities to the peptides. For targeting, the protein or peptide used for conjugation will be selected based on the cell or tissue being targeted for therapy (Lee, R. et al. Arthritis. Rheum. 46: 2109-2120 (2002); Pasqualini, R. Q. J. Nuc. Med. 43: 159-62 (1999); Pasgualini, R. Nature 380: 364-366 (1996); hereby incorporated by reference). For targeting to the central nervous system, suitable carrier proteins include, among others, antibodies against the transferrin receptor (see U.S. Pat. No. 5,527,527, hereby incorporated by reference); cationized albumin; met-enkephalin (see U.S. Patent No. 5,442,043, 4902,505, and 4,801,575; incorporated by reference); and antibodies to human insulin receptor (see Pardridge, W. M. et al. Pharm. Res. 12: 807-816 (1995 ); incorporated by reference). The proteins may also compromise poly-amino acids including, but not limited to, polyarginine; and polylysine, polyaspartic acid, etc., which may be incorporated into other polymers, such as polyethylene glycol, for preparation of vesicles or particles containing the conjugated peptides.

Targeting to the central nervous system is also done by coupling the peptides to conjugates of proteins and small molecules that are readily transported across the blood brain barrier. For instance, anti-transferrin receptor monoclonal antibody OX26 coupled to streptavidin is selectively transported across the blood brain barrier. Consequently, conjugating the subject peptides to this antibody-streptavidin complex allows delivery of the attached peptide into the brain (Boado, et al. J. Pharma. Sci. 87:1308-1315 (1998)).

In another aspect, the subject peptides may be expressed in conjunction with other peptides or proteins, so as to be a portion of the polypeptide chain, either internal, or at the N- or C-terminus to form chimeric proteins or fusion proteins. By “fusion polypeptide” or “fusion protein” or “chimeric protein” herein is meant a protein composed of a plurality of protein components that, while typically joined in the native state, are joined by the respective amino and carboxy termini through a peptide linkage to form a continuous polypeptide. Plurality in this context means at least two, and preferred embodiments generally three to twelve components, although more may be used. It will be appreciated that the protein components can be joined directly or joined through a peptide linker/spacer as outlined below.

Fusion polypeptides may be made to a variety of other peptides or proteins to display the subject peptides in a conformationally restricted form, for targeting to cells and tissues, for targeting to intracellular compartments, tracking the fusion protein in a cell or an organism, and screening for other molecules that bind the peptides. Proteins useful for generating fusion proteins include various reporter proteins, structural proteins, cell surface receptors, receptor ligands, toxins, and enzymes. Exemplary proteins include fluorescent proteins (e.g., Aequoria victoria GFP, Renilla reniformis GFP, Renilla muelleri GFP, luciferases, etc., and variants thereof); β-galactosidase; alkaline phosphatase; E. coli. maltose binding protein; coat proteins of filamentous bacteriophage (e.g., minor coat protein, pIII, or the major coat protein, pVIII, for purposes of phage display).

Fusion proteins also encompass fusions with fragments of proteins or other peptides, either alone or as part of a larger protein sequence. Thus, the fusion polypeptides may comprise fusion partners. By “fusion partners” herein is meant a sequence that is associated with the peptide that confers all members of the proteins in that class a common function or ability. Fusion partners can be heterologous (i.e., not native to the host cell) or synthetic (i.e., not native to any cell). The fusion partners include, but are not limited to, a) presentation structures, which provide the subject peptides in a conformationally restricted or stable form; b) targeting sequences, which allow localization of the peptide to a subcellular or extracellular compartment; c) stability sequences, which affects stability or protection from degradation to the peptide or the nucleic acid encoding it; d) linker sequences, which conformationally decouples the oligopeptide from the fusion partner; and e) any combination of the above.

In one aspect, the fusion partner is a presentation structure. By “presentation structure” as used herein is meant a sequence that when fused to the subject peptides presents the peptides in a conformationally restricted form. Preferred presentation structures enhance binding interactions with other binding partners by presenting a peptide on a solvent exposed exterior surface, such as a loop. Generally, such presentation structures comprise a first portion joined to the N-terminus of the peptide and a second portion joined to the C-terminal end of the subject peptide. That is, the peptide of the present invention is inserted into the presentation structures. Preferably, the presentation structures are selected or designed to have minimal biological activity when expressed in the target cells.

Preferably, the presentation structures maximize accessibility to the peptides by displaying or presenting the peptide on an exterior loop. Suitable presentation structures include, but are not limited to, coiled coil stem structures, minibody structures, loops on β-turns, dimerization sequences, cysteine linked structures, transglutaminase linked structures, cyclic peptides, helical barrels, leucine zipper motifs, etc.

In one embodiment, the presentation structure is a coiled-coil structure, which allows presentation of the subject peptide on an exterior loop (see Myszka et al. Biochemistry 33: 2362-2373 (1994)), such as a coiled-coil leucine zipper domain (see Martin et al. EMBO J. 13: 5303-5309 (1994)). The presentation structure may also comprise minibody structures, which is essentially comprised of a minimal antibody complementarity region. The minibody structure generally provides two peptide regions that are presented along a single face of the tertiary structure in the folded protein (see Bianchi et al. J. Mol Biol. 236: 649-659 (1994); Tramontano et al. J. Mol. Recognit. 7: 9-24 (1994)).

In another aspect, the presentation structure comprises two dimerization sequences. The dimerization sequences, which can be same or different, associate non-covalently with sufficient affinity under physiological conditions to structurally constrain the displayed peptide; that is, if a dimerization sequence is used at each terminus of the subject oligopeptide, the resulting structure can display the subject peptide in a structurally limited form. A variety of sequences are suitable as dimerization sequences (see for example, WO 99/51625; incorporated by reference). Any number of protein-protein interaction sequences known in the art are useful.

In a further aspect, the presentation sequence confers the ability to bind metal ions to generate a conformationally restricted secondary structure. Thus, for example, C2H2 zinc finger sequences are used. C2H2 sequences have two cysteines and two histidines placed such that a zinc ion is chelated. Zinc finger domains are known to occur independently in multiple zinc-finger peptides to form structurally independent, flexibly linked domains (see Nakaseko, Y. et al. J. Mol Biol. 228: 619-636 (1992)). A general consensus sequence is (5 amino acids)-C-(2 to 3 amino acids)-C-(4 to 12 amino acids)-H-(3 amino acids)-H-(5 amino acids). A preferred example would be -FQCEEC-random peptide of 3 to 20 amino acids-HIRSHTG. Similarly, CCHC boxes having a consensus sequence -C-(2 amino acids)-C-(4 to 20 random peptide)-H-(4 amino acids)-C- can be used, (see Bavoso, A. et al. Biochem. Biophys. Res. Commun. 242: 385-389 (1998)). Other examples include (1) -VKCFNC-4 to 20 random amino acids-HTARNCR-, based on the nucleocapsid protein P2; (2) a sequence modified from that of the naturally occurring zinc-binding peptide of the Lasp-1 LIM domain (Hammarstrom, A. et al. Biochemistry 35: 12723-32 (1996)); and (3) -MNPNCARCG4 to 20 random amino acids-HKACF-, based on the NMR structural ensemble 1ZFP (Hammarstrom et al., supra).

In yet another aspect, the presentation structure is a sequence that comprises two or more cysteine residues, such that a disulfide bond may be formed, resulting in a conformationally constrained structure. That is, use of cysteine containing peptide sequences at each terminus of the subject peptides results in cyclic peptide structures, as described above. A cyclic structure reduces susceptibility of the presented peptide to proteolysis and increases accessibility to its target molecules. As will be appreciated by those skilled in the art, this particular embodiment is particularly suited when secretory targeting sequences are used to direct the peptide to the extracellular space.

In another embodiment, the fusion partner is a targeting sequence. Targeting sequences comprise binding sequences capable of causing binding of the expressed product to a predetermined molecule or class of molecules while retaining bioactivity of the expression product; sequences signaling selective degradation of the fusion protein or binding partners; and sequences capable of constitutively localizing peptides to a predetermined cellular locale. Typical cellular locations include subcellular locations (e.g, Golgi, endoplasmic recticulum, nucleus, nucleoli, nuclear membrane, mitochondria, secretory vesicles, lysosomes) and extracellular locations by use of secretory signals.

Various targeting sequences are known in the art. Targeting to nucleus is achieved by use of nuclear localization signals (NLS). NLSs are generally short, positively charged domains that direct the proteins in which the NLS is present to the cell's nucleus. Typical NLS sequences include the single basic NLS of SV40 large T antigen (Kalderon et al. Cell 39: 499-509 (1984)); human retinoic acid receptor-β, nuclear localization signal (NF-kB p50 and p65 (Ghosh et al. Cell 62: 1019-1029 (1990)); Nolan et al. Cell 64: 961-999 (1991)); and the double basic NLS exemplified by nucleoplasmin (Dingwall et al. J. Cell Biol. 107: 641-649 (1988)).

In another aspect the targeting sequences are membrane anchoring sequences. Peptides are directed to the membrane via signal sequences and stably incorporated in the membrane through a hydrophobic transmembrane domain (designated as TM). The TM segment is positioned appropriately on the expressed fusion protein to display the subject peptide either intracellularly or extracellularly, as is known in the art. Membrane anchoring sequences and signal sequences include, but are not limited to, those derived from (a) class I integral membrane proteins such as IL-2 receptor β-chain; Hatekeyama et al. Science 244: 551-556 (1989)) and inuslin receptor β-chain (Hetekayama et al, supra); (b) class II integral membrane proteins such as neutral endopeptidase (Malfroy et al Biochem. Biophys. Res. Commun. 144: 59-66 (1987)); and (c) type III proteins such as human cytochrome P450 NF25 (Hetekayama et al, supra); and those from CD8, ICAM-2, IL-8R, and LFA-1.

Membrane anchoring sequences also include the GPI anchor, which results in covalent bond formation between the GPI anchor sequence and the lipid bilayer via a glycosyl-phosphatidylinositol. GPI anchor sequences are found in various proteins, including Thy-1 and DAF (see Homans et al. Nature 333: 269-272 (1988)). Similarly, acylation sequences allow for attachment of lipid moieties, e.g., isoprenylation (i.e., farnesyl and geranyl-geranyl; see Farnsworth et al. Proc. Natl. Acad. Sci. USA 91: 11963-11967 (1994) and Aronheim et al. Cell 78: 949-61 (1994)), myristoylation (Stickney, J. T. Methods Enzymol. 332: 64-77 (2001)), or palmitoylation. In one aspect, the subject peptide will be bound to a lipid group at a terminus, so as to be able to be bound to a lipid membrane, such as that of a liposome.

Other intracellular targeting sequences are lysozomal targeting sequences (e.g., sequences in LAMP-1 and LAMP-2; Uthayakumar et al. Cell Mol. Biol. Res. 41: 405-420 (1995) and Konecki et al. Biochem. Biophys. Res. Comm. 205:1-5 (1994)); mitochondrial localization sequences (e.g., mitochondrial matrix sequences, mitochondrial inner membrane sequences, mitochondrial intermembrance sequences, or mitochondrial outer membrane sequences; see Shatz, G. Eur. J. Biochem. 165: 1-6 (1987)); endoplasmic recticulum localization sequences (e.g., calreticulin, Pelham, H. R. Royal Soc. London Transactions B: 1-10 (1992); adenovirus E3/19K protein, Jackson et al. EMBO J. 9: 3153-3162 (1990)); and peroxisome localization sequences (e.g., luciferase peroxisome matrix sequence, Keller et al. Proc. Natl. Acad. Sci. USA 4: 3264-3268 (1987)).

In another aspect, the targeting sequence is a secretory signal sequence which effects secretion of the peptide. A large number of secretory sequences are known to direct secretion of a peptide into the extracellular space when placed at the amino end relative to the peptide of interest, particularly for secretion of a peptide by cells, including transplanted cells. Suitable secretory signals included those found in IL-2 (Villinger et al. J. Immuno. 155: 3946-3954 (1995)), growth hormone (Roskam et al. Nucleic Acids Res. 7: 305-320 (1979)), preproinsulin, and influenza HA protein.

The fusion partner may further comprise a stability sequence, which confers stability to the fusion protein or the nucleic acid encoding it. Thus, for example, incorporation of glycines after the initiating methionine (e.g., MG or MGG) can stabilize or protect the fused peptide from degradation via ubiquitination as per the N-End rule of Varshavsky, thus conferring increased half-life in a cell.

Additional amino acids may be added for tagging the peptide for purposes of detection or purification. These sequences may comprise epitopes recognized by antibodies (e.g., flag tags) or sequences that bind ligands, such a metals ions. Various tag sequences and ligand binding sequences are well known in the art. These include, but are not limited to, poly-histidine (e.g., 6×His tags, which are recognized by antibodies but also bind divalent metal ions); poly-histidine-glycine (poly-his-gly) tags; flu HA tag polypeptide; c-myc tag; Flag peptide (Hopp et al. BioTechnology 6: 1204-1210 (1988)); KT3 epitope peptide; tubulin epitope peptide (Skinner et al. J. Biol. Chem. 266: 15163-12166 (1991)); and T7 gene 10 protein peptide tag (Lutz-Freyermuth et al. Proc. Natl. Acad. Sci. USA 87: 6363-6397 (1990)).

Fusion partners include linker or tethering sequences for linking the peptides and for presenting the peptides in an unhindered structure. As discussed above, useful linkers include glycine polymers (G)n where n is 1 to about 7, glycine-serine polymers (e.g., (GS)n, (GSGGS)n and (GGGS)n, where n is at least 1), glycine-alanine polymers, alanine-serine polymers, and other flexible linkers known in the art. Preferably, the linkers are glycine or glycine-serine polymers since these amino acids are relatively unstructured, hydrophilic, and are effective for joining segments of proteins and peptides.

If desired, various groups are introduced into the peptide during synthesis or during expression, which allows for linking to other molecules or to a surface. Thus, cysteines can be used to make thioethers or cyclic peptides, histidines for linking to a metal ion complex, carboxyl groups for forming amides or esters, amino groups for forming amides, and the like. When cysteine residues are introduced for cyclizing the peptide, formation of disulfide bonds are conducted in the presence of mild oxidizing agents. Chemical oxidants may be used, or the cysteine bearing peptides are exposed to oxygen to form the linkages, typically in a suitable solution such as a aqueous buffer containing DMSO. As described above, lipids may be attached either chemically or by use of appropriate lipidation sequences in the expressed peptide.

For conjugating various molecules to the peptides of the present invention, functional groups on the peptides and the other molecule are reacted in the presence of an appropriate conjugating (e.g., crosslinking) agent. The type of conjugating or crosslinking agent used will depend on the functional groups, such as primary amines, sulfhydryls, carbonyls, carbohydrates and carboxylic acids being used. Agents may be fixatives and crosslinking agents, which may be homobifunctional, heterobifunctional, or trifunctional crosslinking agents (Pierce Endogen, Chicago, Ill.). Commonly used fixatives and crosslinking agents include formaldehyde, glutaraldehyde, 1,1-bis(diazoacetyl)-2-phenylethane, N-hydroxysuccinimide esters, dissuccimidyl esters, maleimides (e.g., bis-N-maleimido-1-8-octane), and carbodiimides (e.g., N-ethyl-N′-(3-dimethylaminopropyl )-carbodiimide; dicyclohexylcarbodiimide. Spacer molecules comprising alkyl or substituted alkyl chains with lengths of 2-20 carbons may be used to separate conjugates. Preferably, reactive functional groups on the peptide not selected for modification are protected prior to coupling of the peptide to other reactive molecules to limit undesired side reactions. By “protecting group” as used herein is a molecule bound to a specific functional group which is selectively removable to reexpose the functional group (see Greene, T. W. and Wuts, P. G. M. Protective Groups in Organic Synthesis (3rd ed.), John Wiley & Sons, Inc., New York, 1999). The peptides may be synthesized with protected amino acid precursors or reacted with protecting groups following synthesis but before reacting with crosslinking agent. Conjugations may also be indirect, for example by attaching a biotin moiety, which can be contacted with a compound or molecule which is coupled to streptavidin or avidin.

For peptides that have reduced activity in the conjugated form, the linkage between the peptides and the conjugated compound is chosen to be sufficiently labile to result in cleavage under desired conditions, for example after transport to desired cells or tissues. Biologically labile covalent bonds, e.g., imimo bonds and esters, are well known in the art (see U.S. Pat. No. 5,108,921, hereby incorporated by reference). These modifications permit administration of the peptides in potentially a less active form, which is then activated by cleavage of the labile bond.

In the present invention, combinations of fusion partners may be used. Any number of combinations of presentation structures, targeting sequences, rescue sequences, tag sequences and stability sequences may be used with or without linker sequences.

Peptide Preparation and Salts

The RDP-58, TCR, or HLA peptides of the present invention may be prepared in a number of ways. Chemical synthesis of peptides are well known in the art. Solid phase synthesis is commonly used and various commercial synthetic apparatuses are available, for example automated synthesizers by Applied Biosystems Inc., Foster City, Calif.; Beckman; etc. Solution phase synthetic methods may also be used, although it is less convenient. By using these standard techniques, naturally occurring amino acids may be substituted with unnatural amino acids, particularly D-stereoisomers, and also with amino acids with side chains having different lengths or functionalities. Functional groups for conjugating to small molecules, label moieties, peptides, or proteins, or for purposes of forming cyclized peptides may be introduced into the molecule during chemical synthesis. In addition, small molecules and label moieties may be attached during the synthetic process. Preferably, introduction of the functional groups and conjugation to other molecules minimally affects the structure and function of the subject peptide.

The peptides of the present invention may be present in the form of a salt, generally in a salt form which is pharmaceutically acceptable. These include inorganic salts of sodium, potassium, lithium, ammonium, calcium, magnesium, iron, zinc, copper, manganese, and the like. Various organic salts of the peptide may also be made with, including, but not limited to, acetic acid, propionic acid, pyruvic acid, maleic acid, succinic acid, tartaric acid, citric acid, benozic acid, cinnamic acid, salicylic acid, etc.

Synthesis of the peptides and derivatives thereof may also be carried out by using recombinant techniques. For recombinant production, one may prepare a nucleic acid sequence which encodes a single oligopeptide or preferably a plurality of the subject peptides in tandem with an intervening amino acid or sequence, which allows for cleavage to the single peptide or head to tail dimers. Where methionine or tryptophane is absent, an intervening methionine or tryptophane may be incorporated, which allows for single amino acid cleavage using CNBr or BNPS-Skatole (2-(2-nitrophenylsulfenyl)-3-methyl-3-bromoindolenine), respectively. Alternatively, cleavage is accomplished by use of sequences that are recognized by particular proteases for enzymatic cleavage or sequences that act as self-cleaving sites (e.g., 2A sequences of apthoviruses and cardioviruses; Donnelly, M. L. J. Gen. Virol. 78: 13-21.(1997); Donnelly, M. L. J. Gen. Virol. 82: 1027-41 (2001), hereby incorporated by reference). The subject peptide may also be made as part of a larger peptide, which can be isolated and the oligopeptide obtained by proteolytic cleavage or chemical cleavage. The particular sequence and the manner of preparation will be determined by convenience, economics, purity required, and the like. To prepare these compositions, a gene encoding a particular peptide, protein, or fusion protein is joined to a DNA sequence encoding the peptides of the present invention to form a fusion nucleic acid, which is introduced into an expression vector. Expression of the fusion nucleic acid is under the control of a suitable promoter and other control sequences, as defined below, for expression in a particular host cell or organism (see, Sambrook et al., Molecular Biology: A Laboratory Manual, 3rd Ed., Cold Spring Harbor Laboratories, Cold Spring Harbor, N.Y., 2001; Ausubel et al. Current Protocols in Molecular Biology, John Wiley & Sons, New York, N.Y., 1988, updates up to 2002; incorporated by reference).

Nucleic Acids, Expression Vectors, and Methods of Introduction

When the synthesis or delivery of the peptides is via nucleic acids encoding the subject peptides, the nucleic acids are cloned into expression vectors and introduced into cells or a host. The expression vectors are either self-replicating extrachromosomal vectors or vectors that integrate into the host chromosome, for example vectors based on retroviruses, vectors with site specific recombination sequences, or by homologous recombination. Generally, these vectors include control sequences operably linked to the nucleic acids encoding the peptides. By “control sequences” is meant nucleic acid sequences necessary for expression of the subject peptides in a particular host organism. Thus, control sequences include sequences required for transcription and translation of the nucleic acids, including, but not limited to, promoter sequences, enhancer or transcriptional activator sequences, ribosomal binding sites, transcriptional start and stop sequences; polyadenylation signals; etc.

A variety of promoters are useful in expressing the peptides of the present invention. The promoters may be constitutive, inducible, and/or cell specific and may comprise natural promoters, synthetic promoters (e.g. tTA tetracycline inducible promoters), or hybrids of various promoters. Promoters are chosen based on, among others, the cell or organism in which the proteins are to be expressed, the level of desired expression, and regulation of expression. Suitable promoters are bacterial promoters (e.g., pL I phage promoter, tac promoter, lac lac promoter, etc.); yeast based promoters (e.g., GAL4 promoter, alcohol dehydrogenase promoter, tryptophane synthase promoter, copper inducible CUPI promoter, etc.), plant promoters (e.g., CaMV S35, nopoline synthase promoter, tobacco mosaic virus promoter, etc), insect promoters (e.g., Autographa nuclear polyhedrosis virus, Aedes DNV viral p& and p61, hsp70, etc.), and promoters for expression mammalian cells (e.g., ubiquitin gene promoter, ribosomal gene promoter, β-globin promoter, thymidine kinase promoter, heat shock protein promoters, and ribosomal gene promoters, etc.), and particularly viral promoters, such as cytomegalovirus (CMV) promoter, simian virus (SV40) promoter, and retroviral promoters.

By “operably linked” herein is meant that a nucleic acid is placed into a functional relationship with another nucleic acid. In the present context, operably linked means that the control sequences are positioned relative to the nucleic acid sequence encoding the subject peptides in such a manner that expression of the encoded peptide occurs. The vectors may comprise plasmids or comprise viral vectors, for example retroviral vectors, which are useful delivery systems if the cells are dividing cells, or lentiviral and adenoviral vectors if the cells are non-dividing cells. Particularly preferred are self-inactivating retroviral vectors (SIN vectors), which have inactivated viral promoters at the 3′-LTR, thereby permiting control of expression of heterologous genes by use of non-viral promoters inserted into the viral vector (see for example, Hoffman et al. Proc. Natl. Acad. Sci. USA 93: 5185 (1996). As will be appreciated by those in the art, modifications of the system by pseudotyping allows use of retroviral vectors for all eukaryotic cells, particularly for higher eukaryotes (Morgan, R.A. et al. J. Virol. 67:4712-21 (1993); Yang, Y. et al. Hum. Gene Ther. 6: 1203-13 (1995)).

In addition, the expression vectors also contain a selectable marker gene to allow selection of transformed host cells. Generally, the selection will confer a detectable phenotype that enriches for cells containing the expression vector and further permits differentiation between cells that express and do not express the selection gene. Selection genes are well known in the art and will vary with the host cell used. Suitable selection genes included genes that render the cell resistant to a drug, genes that permit growth in nutritionally deficient media, and reporter genes (e.g. β-galactosidase, fluorescent proteins, glucouronidase, etc.), all of which are well known in the art and available to the skilled artisan.

There are a variety of techniques available for introducing nucleic acids into viable cells. By “introduced” into herein is meant that the nucleic acid enters the cells in a manner suitable for subsequent expression of the nucleic acid. Techniques for introducing the nucleic acids will vary depending on whether the nucleic acid is transferred in vitro into cultured cells or in vivo into the cells of the intended host organism and the type of host organism. Exemplary techniques for introducing the nucleic acids in vitro include the use of liposomes, Lipofectin®, electroporation, microinjection, cell fusion, DEAE dextran, calcium phosphate precipitation, and biolistic particle bombardment. Techniques for transfer in vivo include direct introduction of the nucleic acid, use of viral vectors, typically retroviral vectors, and liposome mediated transfection, such as viral coated liposome mediated transfection. The nucleic acids expressing the peptides of the present invention may exist transiently or stably in the cytoplasm or stably integrate into the chromosome of the host (i.e., through use of standard regulatory sequences, selection markers, etc.). Suitable selection genes and marker genes are used in the expression vectors of the present invention.

In some situations, it is desirable to include an agent that targets the target cells or tissues, such as an antibody specific for a cell surface protein or the target cell, a ligand for a receptor on the target cell, a lipid component on the cell membrane, or a carbohydrate on the cell surface. If liposomes are employed, proteins that bind a cell surface protein which is endocytosed may be used for targeting and/or facilitating uptake. These include as non-limiting examples, capsid proteins or fragments thereof tropic for a particular cell types, antibodies for proteins which undergo internalization (see Wu et al. J. Biol. Chem. 262: 4429-4432 (1987); Wagner et al. Proc. Natl. Acad. Sci. USA 87: 3410-3414 (1990)), and proteins that direct localization (e.g., antibody to transferrin receptor for targeting to brain) or enhance in vivo half-life.

Expression is done in a wide range of host cells that span prokaryotes and eukaryotes, including bacteria, yeast, plants, insects, and animals. The peptides of the present invention may be expressed in, among others, E. coli., Saccharomyces cerevisiae, Saccharomyces pombe, Tobacco or Arabidopsis plants, insect Schneider cells, and mammalian cells, such as COS, CHO, HeLa, and the like, either intracellularly or in a secreted form by fusing the peptides to an appropriate signal peptide. Secretion from the host cell may be done by fusing the DNA encoding the peptide and a DNA encoding a signal peptide. Secretory signals are well known in the art for bacteria, yeast, insects, plants, and mammalian systems. Nucleic acids expressing the peptides may be inserted into cells, for example stem cells for tissue expression or bacteria for gut expression, and the cells transplanted into the host to provide an in vivo source of the peptides.

Purified Peptides

In a preferred embodiment, the RDP-58, TCR and HLA peptides of the present invention may be purified or isolated after synthesis or expression. By “purified” or “isolated” is meant free from the environment in which the peptide is synthesized or expressed and in a form where it can be practically used. Thus purified or isolated is meant that the peptide or its derivative is substantially pure, i.e., more than 90% pure, preferably more than 95% pure, and preferably more than 99% pure. The peptides and derivatives thereof may be purified and isolated by way known to those skilled in the art, depending on other components present in the sample. Standard purification methods include electrophoretic, immunological, and chromatographic techniques, including ion exchange, hydrophobic, affinity, size exclusion, reverse phase HPLC, and chromatofocusing. The proteins may also be purified by selective solubility, for instance in the presence of salts or organic solvents. The degree of purification necessary will vary depending on use of the subject peptides. Thus, in some instances no purification will be necessary.

For the most part, the compositions used will comprise at least 20% by weight of the desired product, more usually at least about 75% by weight, preferably at least about 95% by weight, and usually at least about 99.5% by weight, relative to contaminants related to the method of product preparation, the purification procedure, and its intended use, for example with a pharmaceutical carrier for the purposes of therapeutic treatment. Usually, the percentages will be based upon total protein.

Pharmaceutical Formulations, Dosage Forms, Dosages, and Methods of Administration

subject compositions, either alone or in combination, may be used in vitro, ex vivo, and in vivo depending on the particular application. In accordance, the present invention provides for administering a pharmaceutical composition comprising a pharmaceutically acceptable carrier and a pharmacologically effective amount of one or more of the subject peptides, or suitable salts thereof. The pharmaceutical composition may be formulated as powders, granules, solutions, suspensions, aerosols, solids, pills, tablets, capsules, gels, topical cremes, suppositories, transdermal patches, etc.

As indicated above, pharmaceutically acceptable salts of the peptides is intended to include any art recognized pharmaceutically acceptable salts including organic and inorganic acids and/or bases. Examples of salts include sodium, potassium, lithium, ammonium, calcium, as well as primary, secondary, and tertiary amines, esters of lower hydrocarbons, such as methyl, ethyl, and propyl. Other salts include organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” comprises any of standard pharmaceutically accepted carriers known to those of ordinary skill in the art in formulating pharmaceutical compositions. Thus, the subject peptides, by themselves, such as being present as pharmaceutically acceptable salts, or as conjugates, or nucleic acid vehicles encoding such peptides, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients. Additionally, the formulations may include bactericidal agents, stabilizers, buffers, emulsifiers, preservatives, sweetening agents, lubricants, or the like. If administration is by oral route, the oligopeptides may be protected from degradation by using a suitable enteric coating, or by other suitable protective means, for example internment in a polymer matrix such as microparticles or pH sensitive hydrogels.

Suitable formulations may be found in, among others, Remington's Pharmaceutical Sciences, 17th edition, Mack Publishing Co., Philadelphia, Pa., 1985 and Handbook of Pharmceutical Excipients, 3rd Ed, Kibbe, A. H. ed., Washington D.C., American Pharmaceutical Association, 2000; hereby incorporated by reference in their entirety. The pharmaceutical compositions described herein can be made in a manner well known to those skilled in the art (e.g., by means conventional in the art, including mixing, dissolving, granulating, levigating, emulsifying, encapsulating, entrapping or lyophilizing processes).

Additionally, the peptides may also be introduced or encapsulated into the lumen of liposomes for delivery and for extending life time of the peptide formulations ex vivo or in vivo. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids; and other lipids (see for example, U.S. Pat. No. 5,833,948).

Cationic lipids are also suitable for forming liposomes. Generally, the cationic lipids have a net positive charge and have a lipophilic portion, such as a sterol or an acyl or diacyl side chain. Preferably, the head group is positively charged. Typical cationic lipids include 1,2-dioleyloxy-3-(trimethylamino)propane; N-[1-(2,3,-ditetradecycloxy)propyl]-N,N-dimethyl-N-N-hydroxyethylammonium bromide; N-[1-(2,3-dioleyloxy)propyl]-N,N-dimethyl-N-hydroxy ethylammonium bromide; N-[1-(2,3-dioleyloxy) propyl]-N,N,N-trimethylammonium chloride; 3-[N-(N′,N′-dimethylaminoethane) carbamoyl] cholesterol; and dimethyidioctadecylammonium.

Of particular interest are fusogenic liposomes, which are characterized by their ability to fuse with a cell membrane upon appropriate change in physiological condition or by presence of fusogenic component, particularly a fusogenic peptide or protein. In one aspect, the fusogenic liposomes are pH and temperature sensitive in that fusion with a cell membrane is affected by change in temperature and/or pH (see for example, U.S. Pat. No. 4,789,633 and 4,873,089). Generally, pH sensitive liposomes are acid sensitive. Thus, fusion is enhanced in physiological environments where the pH is mildly acidic, for example the environment of a lysosome, endosome and inflammatory tissues. This property allows direct release of the liposome contents into the intracellular environment following endocytosis of liposomes (see Mizoue, T. Int. J. Pharm. 237: 129-137 (2002)).

Another form of fusogenic liposomes comprise liposomes that contain a fusion enhancing agent. That is, when incorporated into the liposome or attached to the lipids, the agents enhance fusion of the liposome with other cellular membranes, thus resulting in delivery of the liposome contents into the cell. The agents may be fusion enhancing peptides or proteins, including hemaggulutinin HA2 of influenza virus (Schoen, P. Gene Ther. 6: 823-832 (1999)); Sendai virus envelope glycoproteins (Mizuguchi, H. Biochem. Biophys. Res. Commun. 218: 402-407 (1996)); vesicular stomatitis virus envelope glycoproteins (VSV-G) glycoprotein (Abe, A. et al. J Virol 72: 6159-63 (1998)); peptide segments or mimics of fusion enhancing proteins; and synthetic fusion enhancing peptides (Kono, K. et al. Biochim. Biophys. Acta. 1164: 81-90 (1993); Pecheur, E. I. Biochemistry 37: 2361-71 (1998); U.S. Pat. No. 6,372,720).

Liposomes also include vesicles derivatized with a hydrophilic polymer, as provided in U.S. Pat. No. 5,013,556 and 5,395,619, hereby incorporated by reference, (see also, Kono, K. et al. J. Controlled Release 68: 225-35 (2000); Zalipsky, S. et al. Bioconjug. Chem. 6: 705-708 (1995)) to extend the circulation lifetime in vivo. Hydrophilic polymers for coating or derivation of the liposomes include polyethylene glycol, polyvinylpyrrolidone, polyvinylmethyl ether, polyaspartamide, hydroxymethyl cellulose, hydroxyethyl cellulose, and the like. In addition, as described above, attaching proteins that bind a cell surface protein which is endocytosed, e.g., capsid proteins or fragments thereof tropic for a particular cell types and antibodies for cell surface proteins which undergo internalization (see Wu et al, supra; Wagner et al., supra), may be used for targeting and/or facilitating uptake of the liposomes to specific cells or tissues.

Liposomes are prepared by ways well known in the art (see for example, Szoka, F. et al. Ann. Rev. Biophys. Bioeng. 9: 467-508 (1980)). One typical method is the lipid film hydration technique in which lipid components are mixed in an organic solvent followed by evaporation of the solvent to generate a lipid film. Hydration of the film in aqueous buffer solution, preferably containing the subject peptide or nucleic acid, results in an emulsion, which is sonicated or extruded to reduce the size and polydispersity. Other methods include reverse-phase evaporation (see Pidgeon, C. et al. Biochemistry 26: 17-29 (1987); Duzgunes, N. et al. Biochim. Biophys. Acta. 732: 289-99 (1983)), freezing and thawing of phospholipid mixtures, and ether infusion.

In another preferred embodiment, the carriers are in the form of microparticles, microcapsules, micropheres and nanoparticles, which may be biodegradable or non-biodegradable (see for example, Microencapsulates: Methods and Industrial Applications, Drugs and Phamaceutical Sciences, Vol 73, Benita, S. ed, Marcel Dekker Inc., New York, 1996; incorporated by reference). As used herein, microparticles, microspheres, microcapsules and nanoparticles mean a particle, which is typically a solid, containing the substance to be delivered. The substance is within the core of the particle or attached to the particle's polymer network. Generally, the difference between microparticles (or microcapsules or microspheres) and nanoparticles is one of size. As used herein, microparticles have a particle size range of about 1 to about >1000 microns. Nanoparticles have a particle size range of about 10 to about 1000 nm.

A variety of materials are useful for making microparticles. Non-biodegradable microcapsules and microparticles include, but not limited to, those made of polysulfones, poly(acrylonitrile-co-vinyl chloride), ethylene-vinyl acetate, hydroxyethylmethacrylate-methyl-methacrylate copolymers. These are useful for implantation purposes where the encapsulated peptide diffuses out from the capsules. In another aspect, the microcapsules and microparticles are based on biodegradable polymers, preferably those that display low toxicity and are well tolerated by the immune system. These include protein based microcapsulates and microparticles made from fibrin, casein, serum albumin, collagen, gelatin, lecithin, chitosan, alginate or poly-amino acids such as poly-lysine. Biodegradable synthetic polymers for encapsulating may comprise polymers such as polylactide (PLA), polyglycolide (PGA), poly(lactide-co-glycolide) (PLGA), poly(caprolactone), polydioxanone trimethylene carbonate, polyhybroxyalkonates (e.g., poly(β-hydroxybutyrate)), poly(β-ethyl glutamate), poly(DTH iminocarbony (bisphenol A iminocarbonate), poly (ortho ester), and polycyanoacrylate. Various methods for making microparticles containing the subject compositions are well known in the art, including solvent removal process (see for example, U.S. Pat. No. 4,389,330); emulsification and evaporation (Maysinger, D. et al. Exp. Neuro. 141: 47-56 (1996); Jeffrey, H. et al. Pharm. Res. 10: 362-68 (1993)), spray drying, and extrusion methods.

Another type of carrier is nanoparticles, which are generally suitable for intravenous administrations. Submicron and nanoparticles are generally made from amphiphilic diblock, triblock, or multiblock copolymers as is known in the art. Polymers useful in forming nanoparticles include, but are limited to, poly(lactic acid) (PLA; see Zambaux et al., J. Control Release 60: 179-188 (1999)), poly(lactide-co-glycolide), blends of poly(lactide-co-glycolide) and polycarprolactone, diblock polymer poly(l-leucine-block-l-glutamate), diblock and triblock poly(lactic acid) (PLA) and poly(ethylene oxide) (PEO) (see De Jaeghere, F. et al., Pharm. Dev. Technol. ;5: 473-83 (2000)), acrylates, arylamides, polystyrene, and the like. As described for microparticles, nanoparticles may be non-biodegradable or biodegradeable. Nanoparticles may be also be made from poly(alkylcyanoacrylate), for example poly(butylcyanoacrylate), in which the peptide is absorbed onto the nanoparticles and coated with surfactants (e.g., polysorbate 80). Methods for making nanoparticles are similar to those for making microparticles and include, among others, emulsion polymerization in continuous aqueous phase, emulsification-evaporation, solvent displacement, and emulsification-diffusion techniques (see Kreuter, J. Nano-particle Preparation and Applications, In Microcapsules and nanoparticles in medicine and pharmacy,” (M. Donbrow, ed.), pg. 125-148, CRC Press, Boca Rotan, Fla., 1991; incorporated by reference).

Hydrogels are also useful in delivering the subject agents into a host. Generally, hydrogels are crosslinked, hydrophilic polymer networks permeable to a wide variety of drug compounds, including peptides. Hydrogels have the advantage of selective trigger of polymer swelling, which results in controlled release of the entrapped drug compound. Depending on the composition of the polymer network, swelling and subsequent release may be triggered by a variety of stimuli, including pH, ionic strength, thermal, electrical, ultrasound, and enzyme activities. Non-limiting examples of polymers useful in hydrogel compositions include, among others, those formed from polymers of poly(lactide-co-glycolide), poly(N-isopropylacrylamide); poly(methacrylic acid-g-polyethylene glycol); polyacrylic acid and poly(oxypropylene-co-oxyethylene) glycol; and natural compounds such as chrondroitan sulfate, chitosan, gelatin, or mixtures of synthetic and natural polymers, for example chitosan-poly(ethylene oxide). The polymers are crosslinked reversibly or irreversibly to form gels embedded with the oligopeptides of the present invention (see for example, U.S. Pat. No. 6,451,346; 6,410,645; 6,432,440; 6,395,299; 6,361,797; 6,333,194; 6,297,337 Johnson, O. et al., Nature Med. 2: 795 (1996); incorporated by reference in their entirety).

In one preferred embodiment, the gel polymers are acrylic acid polymers, preferably carbomers (e.g., carboxypolymethylene), such as Carbopol (e.g., Carbopol 420-430, 475, 488, 493, 910, 934P, 974P, and the like; Brock et al., Pharmacotherapy 14: 430-437 (1994)), which are non-linear polymers of acrylic acid crosslinked with polyalkenyl polyether. Others types of carbomers include acrylic acids crosslinked with polyfunctional compounds, such as polyallysucrose. In addition to the advantage of hydrating and swelling to a gel, which entraps the subject compounds and limits their release, carbomer gels are mucoadhesive. The polymers adheres to the intestinal mucosal membrane, thus resulting in local delivery of the peptides (see Hutton et al. Clin. Sci. 78: 265-271 (1990); Pullan et al., Gut 34: 676-679 (1993), hereby incorporated by reference). In addition, these polymers have the added advantage of limiting intestinal protease activity.

The concentrations of the peptides or nucleic acid encoding therefore will be determined empirically in accordance with conventional procedures for the particular purpose. Generally, for administering the peptides ex vivo or in vivo for therapeutic purposes, the subject formulations are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms of the disorder or disease.

The amount administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but are not limited to, size and weight of the subject, the age and sex of the subject, the severity of the symptom, the stage of the disease, method of delivery of the agent, half-life of the agents, and efficacy of the agents. Stage of the disease to consider include whether the disease is acute or chronic, relapsing or remitting phase, and the progressiveness of the disease. Determining the dosages and times of administration for a therapeutically effective amount are well within the skill of the ordinary person in the art.

The toxicity and therapeutic efficacy are generally determined by cell culture assays and/or experimental animals, typically by determining a LD₅₀ (lethal dose to 50% of the test population) and ED₅ (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the detailed disclosure provided herein.

Generally, in the case where formulations are administered directly to a host, the present invention provides for a bolus or infusion of the subject composition that will be administered in the range of about 0.1-50, more usually from about 1-25 mg/kg body weight of host. The amount will generally be adjusted depending upon the half-life of the peptide where the half life will generally be at least one minute, more usually at least about 10 min, desirably in the range of about 10 min to 12 h. Short half-lives are acceptable, so long as efficacy can be achieved with individual dosages, continuous infusion, or repetitive dosages. Formulations for administration may be presented in unit a dosage form, e.g., in ampules, capsules, pills, or in multidose containers or injectables.

Dosages in the lower portion of the range and even lower dosages may be employed, where the peptide has an enhanced half-life or is provided as a depot, such as a slow release composition comprising particles, a polymer matrix which maintains the peptide over an extended period of time (e.g., a collagen matrix, carbomer, etc.), use of a pump which continuously infuses the peptide over an extended period of time with a substantially continuous rate, or the like. The host or subject may be any mammal including domestic animals, pets, laboratory animals, primates, particularly humans subjects.

In addition to administering the subject peptide compositions directly to a cell culture in vitro, to particular cells ex vivo, or to a mammalian host in vivo, nucleic acid molecules (DNA or RNA) encoding the subject peptides may also be administered thereto, thereby providing an effective source of the subject peptides for the application desired. As described above, nucleic acid molecules encoding the subject peptides may be cloned into any of a number of well known expression plasmids (see Sambrook et al., supra) and/or viral vectors, preferably adenoviral or retroviral vectors (see for example, Jacobs et al., J. Viro. 66:2086-2095 (1992), Lowenstein, Bio/Technology 12:1075-1079 (1994) and Berkner, Biotechniques 6:616-624 (1988)), under the transcriptional regulation of control sequences which function to promote expression of the nucleic acid in the appropriate environment. Such nucleic acid-based vehicles may be administered directly to the cells or tissues ex vivo (e.g., ex vivo viral infection of cells for transplant of peptide producing cells) or to a desired site in vivo, e.g. by injection, catheter, orally (e.g., hybrogels), and the like, or, in the case of viral-based vectors, by systemic administration. Tissue specific promoters may optionally be employed, assuring that the peptide of interest is expressed only in a particular tissue or cell type of choice. Methods for recombinantly preparing such nucleic acid-based vehicles are well known in the art, as are techniques for administering nucleic acid-based vehicles for peptide production.

For the purposes of this invention, the methods of administration is chosen depending on the condition being treated, the form of the subject compositions, and the pharmaceutical composition. Administration of the oligopeptides can be done in a variety of ways, including, but not limited to, cutaneously, subcutaneously, intravenously, orally, topically, transdermally, intraperitoneally, intramuscularly, nasally, and rectally (e.g., colonic administration). For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, oral administration can be accompanied by rectal or topical administration to the affected area. Alternatively, oral administration is used in conjunction with intravenous or parenteral injections.

When the subject peptides are used to treat neurological disorders, the compositions are administered by routes and methods resulting in exposure of the afflicted neuronal tissue and cells to the subject peptides. This consideration is especially important in treating the central nervous system because of the blood-brain barrier (BBB), which limits delivery of therapeutic compounds into the brain. In demyelinating diseases, the compromised state of the blood-brain barrier may allow delivery of active agents by systemic administration (e.g., subcutaneous, intravenous, or oral). Where a more directed delivery is beneficial or required, methods for delivering the subject peptides into the CNS may be used. The method of administration may involve direct infusion into the cerebrospinal fluid via intrathecal or intraventricular route or implantation into the CNS area. Direct intracerebral infusion into particular neuronal populations is also contemplated. For example, see Gill et al., Nat. Med., 9:589-595, 2003. In another embodiment, the peptides are coupled to a drug transporter or carriers, as described above, which permit transport across the blood-brain barrier (see also, Bickel, U. Adv. Drug Deliv. Rev. 46: 247-79 (2001)). Drug transporters and carriers useful for this purpose include lipids, cationized albumin, transferrin receptor antibody, liposomes, microparticles, or nanoparticles. These carriers undergo absorptive uptake or internalization by receptor mediated endocytosis, resulting in passage across the blood brain barrier. Conjugating avidin to the carriers or directly to the oligopeptide allows absorptive-mediated endocytosis of the conjugate, thus providing a useful method for drug delivery. These formulations allow systemic administration of the peptides while targeting damaging immune reactions in the nervous system. Alternatively, the conjugates and carriers containing the subject peptides may be delivered directly to the CNS.

Delivery of the peptides to the CNS may also rely on disruptions to the blood brain barrier, such as intracranial infusion with hypertonic mannitol solutions. Alternatively, it may be preferable to administer the peptide in combination with agents that increase transport across the blood brain barrier. These compounds have the effect of increasing permeability across the blood brain barrier and may or may not be conjugated to the subject peptides. These agents include, but are not limited to, bradykinin and agonist derivatives (U.S. Pat. No. 5,112,596, incorporated by reference) and receptor mediated permeabilizers (A7; U.S. Pat. Nos. 5,268,164 and 5,506,206, incorporated by reference). The solution is introduced intravenously (e.g., via the carotid artery) or by other acceptable routes. Concommitant with or subsequent to disruption, the pharmaceutically acceptable carriers, for example nanoparticles, liposome encapsulated peptides, or genetically engineered retroviruses, are introduced into the host to deliver the peptides to the brain.

Administration of a pharmaceutically effective amount to the brain may also be achieved through the olfactory neural pathway, as provided in U.S. Pat. No. 6,342,478, hereby incorporated by reference. Delivery of the subject peptides via the olfactory system in a pharmaceutically acceptable carrier bypasses the blood brain barrier to permit delivery of the agents directly to the brain. Since there is no significant dilution of the oligpeptides by physiological fluids, concentrated delivery of subject peptides are possible. Administration is done by intranasal application of the subject peptides in a suitable carrier in the form of drops, spray, or powder.

The delivery systems also include sustained release or long term delivery methods, which are well known to those skilled in the art. By “sustained release or” “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the subject peptide, such as biodegradable polymers described above; pumps, including peristaltic pumps and fluorocarbon propellant pumps; osmotic and mini-osmotic pumps; and the like. Peristaltic pumps deliver a set amount of drug with each activation of the pump, and the reservoir can be refilled, preferably percutaneously through a port. A controller sets the dosage and can also provides a readout on dosage delivered, dosage remaining, and frequency of delivery. Fluorocarbon propellant pumps utilize a fluorocarbon liquid to operate the pump. The fluorocarbon liquid exerts a vapor pressure above atmospheric pressure and compresses a chamber containing the drug to release the drug. Osmotic pumps (and mini-osmotic pumps) utilize osmotic pressure to release the drug at a constant rate. The drug is contained in an impermeable diaphragm, which is surrounded by the osmotic agent. A semipermeable membrane contains the osmotic agent, and the entire pump is housed in a casing. Diffusion of water through the semipermeable membrane squeezes the diaphragm holding the drug, forcing the drug into bloodstream, organ, or tissue. These and other such implants are particularly useful in treating a disease condition, especially those manifesting recurring episodes or which are progressive in nature, by delivering the oligopeptides of the invention via systemic (e.g., intravenous or subcutaneous) or localized doses (e.g., intracerebroventricular) in a sustained, long term manner.

In one preferred embodiment, the method of administration is by oral delivery, in the form of a powder, tablet, pill, or capsule. Pharmaceutical formulations for oral administration may be made by combining one or more peptide with suitable excipients, such as sugars (e.g., lactose, sucrose, mannitol, or sorbitol), cellulose (e.g., starch, methyl cellulose, hydroxylmethyl cellulose, carbonxymethyl cellulose, etc.), gelatin, glycine, saccharin, magnesium carbonate, calcium carbonate, polymers such as polyethylene glycol or polyvinylpyrrolidone, and the like. The pills, tablets, or capsules may have an enteric coating, which remains intact in the stomach but dissolves in the intestine. Various enteric coating are known in the art, a number of which are commercially available, including, but not limited to, methacrylic acid-methacrylic acid ester copolymers, polymer cellulose ether, cellulose acetate phathalate, polyvinyl acetate phthalate, hydroxypropyl methyl cellulose phthalate, and the like. Alternatively, oral formulations of the peptides are in prepared in a suitable diluent. Suitable diluents include various liquid form (e.g., syrups, slurries, suspensions, etc.) in aqueous diluents such as water, saline, phosphate buffered saline, aqueous ethanol, solutions of sugars (e.g. sucrose, mannitol, or sorbitol), glycerol, aqueous suspensions of gelatin, methyl cellulose, hydroxylmethyl cellulose, cyclodextrins, and the like. As used herein, diluent or aqueous solutions also include infant formula. In some embodiments, lipohilic solvents are used, including oils, for instance vegetable oils, peanut oil, sesame oil, olive oil, corn oil, safflower oil, soybean oil, etc.); fatty acid esters, such as oleates, triglycerides, etc.; cholesterol derivatives, including cholesterol oleate, cholesterol linoleate, cholesterol myristilate, etc.; liposomes; and the like.

In one embodiment, administration is done rectally. This may use formulations suitable for topical application in the form of salves, tinctures, cremes, or for application into the lumen of the intestine by use of compositions in the form of suppositories, enemas, foams, etc. Suppositories may contain conventional suppository bases such as cocoa butter, carbowaxes, polyethylene glycols, or glycerides, which are solid or semi-solid at room temperature but liquid at body temperature.

In yet another preferred embodiment, the administration is carried out cutaneously, subcutaneously, intraperitonealy, intramuscularly or intravenously. As discussed above, these are in the form of peptides dissolved or suspended in suitable aqueous medium, as discussed above. Additionally, the pharmaceutical compositions for injection may be prepared in lipophilic solvents, which include, but is not limited to, oils, such as vegetable oils, olive oil, peanut oil, palm oil soybean oil, safflower oil, etc; synthetic fatty acid esters, such as ethyl oleate or triglycerides; cholesterol derivatives, including cholesterol oleate, cholesterol linoleate, cholesterol myristilate, etc.; or liposomes, as described above. The compositions may be prepared directly in the lipophilic solvent or preferably, as oil/water emulsions, (see for example, Liu, F. et al. Pharm. Res. 12: 1060-1064 (1995); Prankerd, R.J. J. Parent Sci. Tech. 44: 13949 (1990); U.S. Pat. No. 5,651,991).

The delivery systems also include sustained release or long term delivery methods, which are well known to those skilled in the art. By “sustained release or” “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the subject peptide, such as biodegradable polymers described above; pumps, including peristaltic pumps and fluorocarbon propellant pumps; osmotic and mini-osmotic pumps; and the like. Peristaltic pumps deliver a set amount of drug with each activation of the pump, and the reservoir can be refilled, preferably percutaneously through a port. A controller sets the dosage and can also provides a readout on dosage delivered, dosage remaining, and frequency of delivery. Fluorocarbon propellant pumps utilize a fluorocarbon liquid to operate the pump. The fluorocarbon liquid exerts a vapor pressure above atmospheric pressure and compresses a chamber containing the drug to release the drug. Osmotic pumps (and mini-osmotic pumps) utilize osmotic pressure to release the drug at a constant rate. The drug is contained in an impermeable diaphragm, which is surrounded by the osmotic agent. A semipermeable membrane contains the osmotic agent, and the entire pump is housed in a casing. Diffusion of water through the semipermeable membrane squeezes the diaphragm holding the drug, forcing the drug into bloodstream, organ, or tissue. These and other such implants are particularly useful in treating an inflammatory disease condition, especially those manifesting recurring episodes or which are progressive in nature, by delivering the oligopeptides of the invention via systemic (e.g., intravenous or subcutaneous) or localized doses in a sustained, long term manner.

present invention also encompasses the therapeutic combinations disclosed herein in the form of a kit or packaged formulation. A kit or packaged formulation as used herein includes one or more dosages of an RDP-58 peptide, and salts thereof, in a container holding the dosages together with instructions for simultaneous or sequential administration to a patient. For example, the package may contain the peptides along with a pharmaceutical carrier combined in the form of a powder for mixing in an aqueous solution, which can be ingested by the afflicted subject. Another example of packaged drug is a preloaded pressure syringe, so that the compositions may be delivered colonically. The package or kit includes appropriate instructions, which encompasses diagrams, recordings (e.g., audio, video, compact disc), and computer programs providing directions for use of the combination therapy.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The examples below additionally illustrate the invention.

All publications and patent applications mentioned in this specification are herein incorporated by reference to the same extent as if each individual publication or patent application was specifically and individually indicated to be incorporated by reference.

### EXPERIMENTAL

### Example 1

RDP-58 Composition Ameliorat s Morphological and Clinical Symptoms in the Acute Lewis Rat Model of Experimental Autoimmune Encephalomyelitis

Experimental Animals

Female Lewis rats (Harlan, Hollister, Calif.) were maintained in the animal facility at Sangstat Medical Corporation under conventional conditions with laboratory chow and water accessible ad libitum. Animals were housed three or four per cage for at least one week prior to study and included in experiments at 13-15 weeks of age. All animal procedures were conducted in complete compliance with the NIH Guide for the Care and Use of Laboratory Animals and approved by Sangstat Medical Corporation IACUC. Any animal deemed to be in a moribund state during experimental proceedings was immediately euthanized according to NIH guidelines.

EAE Induction

For active induction of EAE, rats were immunized with 0.1 ml intradermal injections of encephalitogenic inoculum into each hind footpad (0.2 ml total volume). The inoculum was prepared by emulsifying equal volumes of a 2.5 mg/ml solution of guinea pig myelin basic protein (Sigma Chemical, St. Louis, Mo.) in phosphate buffered saline (PBS) with complete Freund's adjuvant prepared with 4 mg Mycobacterium tuberculosis (Difco, Detroit, Mich) per ml of Incomplete Freund's adjuvant (Sigma).

RDP58 Treatment Procedure

RDP58 was given on different days post-inoculation and at different concentrations to determine optimum dose and time of administration. RDP58 was synthesized by UCB Bioproducts (Belgium) and freshly prepared in a 5% mannitol/sterile water solution for each experiment. In general, animals (n=5-10 per treatment group) were anesthetized (Nembutal, 50 mg/kg) and administered intracerebroventricular (icv) injections of either RDP58 or mannitol alone into the lateral ventricle (coordinates relative to bregma: AP=−0.5, L=1.4, and V=4.0). For timecourse experiments, animals received icv administration of 150 μg RDP58 on day 1, 4, 7, or 10 post-immunization. In the dose-response studies, animals received icv injections of 5, 15, and 50 μg of RDP58 ten days after immunization. A 50 μg RDP58 dose given the day prior to onset was subsequently used in experiments aimed at measuring cytokine response levels.

Evaluation of Clinical Signs

Rats were examined daily for clinical symptoms. Body weight measurements were recorded from day 9 to day 20 and clinical scores were assigned to the animals according to severity of paralysis. Clinical disease was scored using a predetermined scale from 0 to 4 as follows: 0, unaffected; 1, flaccid tail; 2, hind limb paralysis; 3, hind & front limb paralysis; 4, moribund state or death. Several parameters of disease were examined to evaluate the severity of EAE and the efficacy of RDP58 therapy, including mean clinical score, incidence, mean day of onset, disease index, mean maximum severity, and mortality.

Histological analysis

For histological evidence of EAE, rats from each treatment group were sacrificed at the peak of clinical disease on day 13 post-inoculation. Animals were deeply anesthetized with Nembutal and perfused with 4% paraformaldehyde. Brain and intact spinal column were removed, fixed in 4% paraformaldehyde, and embedded in paraffin for sectioning. A series of spinal cord cross sections were prepared for evaluation of lymphocyte infiltration using hematoxylin and eosin (H&E) stain. Subsequent analysis of stained tissue sections was done in a blinded fashion by trained investigators. All sections were examined using standard bright-field optics. The severity of infiltration was expressed as number of perivascular infiltrates per section.

Cytokine mRNA Detection by RT-PCR

Sample Preparation

Animals were sacrificed for molecular analysis at post onset day 5. Brains and spinal cords were removed and frozen in liquid nitrogen, then stored at −80° C. until ready for use. The tissues were subsequently homogenized in 700 μl of a guanidinium isothiocyanate solution containing 49 μl β-mercaptoethanol using an Ultraturrax tissue homogenizer (Jahnke and Kunkel, Staufen i. Breisgau, Germany). Total RNA was extracted by using the Absolutely RNA™ RT-PCR Miniprep Kit (Stratagene, La Jolla, USA) and its quality and quantity was determined with the Agilent 2100 Bioanalyzer System (Agilent Technologies, Palo Alto, USA).

For cDNA synthesis a master solution was prepared by mixing 2 μl odT-Primer (0,1 mg/ml), 8 μl of 5× first strand buffer (GibcoBRL, Paisley, U.K.), 4 μl of dithiothreitol (0.1 M) (GibcoBRL), 4 μl of dNTP (Pharmacia Biotech, Uppsala, Sweden) (2.5 mM), 0.5 μl of RNasin ribonuclease inhibitor (Promega, Madison, USA) (40 U/μl) and 2 μl of RQ1 RNase-free DNAse (Ambion, Austin, USA) (2 U/μl). About 2 μg of total RNA dissolved in DEPC water were reverse transcribed. The mixture was incubated at 37° C. for 30 min, thereafter for 5 min at 75° C. to inactivate the DNAse. The reverse transcription reaction was started by adding 1 μ1 of RNasin ribonuclease inhibitor (40 U/μl) and 1 μl of MMLV-reverse transcriptase (GibcoBRL) (20 U/μl). The mixture was incubated at 42° C. for 1 h and then the reaction was stopped by incubating at 95° C. for 10 min.

Quantification of Cytokine Genes

To measure cytokine mRNA levels, the expression of each gene transcript was analyzed by real-time PCR using the ABI PRISM 7700 Sequence Detection System (TaqMan™, Perkin-Elmer Biosystems, Weiterstadt, Germany). Genes for the following products were investigated: CD3, CD25, INFg, TNFα, IL-10, IL4, iNOS and HO-1. The cycle number at which the amplification plot crosses a fixed theshold above baseline is defined as the threshold cycle (Ct). To control for variation in DNA content across the preparations, all results are normalized to the expression of beta actin. All primers and probes were designed and validated at the Institute of Medical Immunology, Humboldt University, Charité, Berlin.

The PCR reaction was performed in a final volume of 25 μl containing 1 μl cDNA, 12.5 μl Master Mix (TaqMan™ Universal PCR Master Mix, Perkin Elmer, Applied Biosystems, Weiterstadt, Germany), 1 μl fluorogenic hybridization probe, 6 μl primer mix, and 5.5 μl distilled water. After an initial step of 2 min at 50° C. involving activation of uracyl-n-glycosylase and degradation of any pre-existing contaminating RNA sequences, a denaturation and a hot start for AmpliTaq™ Gold DNA polymerase (Perkin Elmer Biosystems) was performed at 95° C. for 10 min. The amplification took place in a two-step PCR cycle including a 15 s denaturation step at 95° C. and 1 min annealing/extension step at 60° C. repeated over 40 cycles. The mean Ct values for beta-actin and the cytokines were calculated from duplicate reactions. Samples were considered negative if the Ct values exceeded 40 cycles

Assays for Cytokine Protein Levels

Cytokine protein levels were examined by either ELISA or a more sensitive bioassay. Brain and spinal cord samples were prepared by homogenization in ice-cold PBS (Sigma) supplemented with protease inhibitor cocktail (Sigma) using a tissue-tearor homogenizor (Stratagene, Cedar Creek, Tex.). The samples were then centrifuged for 15 min at 4,000 rpm to separate extracellular supernatant from the cell pellet. Aliquots of supernatant were prepared and stored at −80° C. until cytokines assays were performed. Levels of IL-6, IL-10, IL-12 were assayed using ELISA kits from Biosource (Camarillo, Calif.). IL-2, IL4, and IFN-g amounts were determined using ELISA Duoset kits from R&D Systems (Minneapolis, Minn.). Cytokine content was expressed as pg/mg total protein. Total protein was determined using the Pierce BCA assay.

Rat TNFa content in homogenates was determined using a bioassay based on cytotoxicity in the L929 cell line (ATCC, Manassas, Va.; CCL-1). This assay relies on quantitation by crystal violet staining of murine L929 fibroblasts, which is an indicator of cell viability. Samples are added to L929 cell monolayers in the presence of 1 mg/ml actinomycin D for 18 hr at 37 oC. Crystal violet (Sigma) at 0.5% is then added to L929 cells for 15 min at room temperature and solubilized with 33% acetic acid. Rat recombinant TNFa (R&D Systems) is included in each assay as standard control. Absorbance is subsequently measured at 570 nm and sample TNF levels calculated from a standard curve.

### Example 2

RDP-58 Prevents Neuronal Cell Death

NGF-dependent neurons are maintained in vitro in media containing NGF. The cells are washed and the media containing NGF is replaced with media lacking NGF and containing anti-NGF antibody. One sample is incubated in the presence of RDP-58, the other in the absence of RDP-58. Apoptosis is measured in the two samples, for example, by TUNEL labeling. RDP-58 inhibits neuronal apoptosis in NGF-dependent neurons induced by NGF deprivation.

We claim:

1. A method for treating a neurological disorder, comprising administering to a patient suffering from said neurological disorder a therapeutically effective amount of an RDP-58 composition.

2. The method according to claim 1, wherein said neurological disorder is an acute neurological disorder.

3. The method according to claim 2, wherein said acute disorder involves inflammation.

4. The method according to claim 3, wherein said acute disorder involving inflammation is selected from the group consisting of hemorrhagic stroke, ischemic stroke, traumatic brain injury, traumatic spinal cord injury, and traumatic peripheral nerve injury.

5. The method according to claim 1, wherein said neurological disorder is a chronic disorder.

6. The method according to claim 5, wherein said chronic disorder involves inflammation.

7. The method according to claim 6, wherein said chronic disorder involving inflammation is a neuromuscular disorder.

8. The method according to claim 7, wherein said neuromuscular disorder is myasthenia gravis.

9. The method according to claim 6, wherein said chronic disorder involving inflammation is HIV-associated dementia.

10. The method according to claim 6, wherein said chronic disorder involving inflammation is a demyelinating disease.

11. The method according to claim 10, wherein said demyelinating disease is selected from the group consisting of multiple sclerosis, acute disseminated encephalomyelitis, optic neuromyelitis, transverse myelopathy, chronic inflammatory demyelinating polyneuropathy (CIDP), and Guillain-Barre syndrome.

12. The method according to claim 6, wherein said chronic disorder involving inflammation is chronic fatigue syndrome.

13. A method for inhibiting neural cell death, comprising contacting at least one neural cell with a neuroprotective amount of an RDP-58 composition.

14. The method according to claim 13, wherein said neural cell is a neuronal cell.

15. The method according to claim 13, wherein said neural cell is a glial cell.

16. The method according to claim 13, wherein said contacting occurs in vitro.

17. The method according to claim 13, wherein said contacting occurs in vivo.

18. The method according to claim 13, wherein said contacting occurs ex vivo.

19. A method for reducing neural cell death in a patient suffering from a neurological disorder, comprising administering to said patient a neuroprotective amount of an RDP58 composition.

20. A method for reducing neural cell death and inflammation in a patient suffering from a neurological disorder, comprising administering to said patient a neuroprotective amount of an RDP-58 composition.

21. A pharmaceutical composition useful for the treatment of a neurological disorder, comprising an RDP-58 composition.

22. The pharmaceutical composition according to claim 21, additionally comprising an agent that is a JNK or p38 inhibitor other than an RDP-58 peptide, a TCR peptide, or an HLA peptide.

23. The pharmaceutical composition according to claim 22, wherein said agent is selected from the group consisting of minocycline, VX-608, SB203580, CEP-1347, SB-202190 and PD169316.

23. The pharmaceutical composition according to claim 21, additionally comprising a neurotrophic factor.

24. The pharmaceutical composition according to claim 23, wherein the neurotrophic factor is selected from the group consisting of GDNF, BDNF, NGF, CNTF, IGF and LIF.

25. A method for increasing the survival of a cell transplanted into a patient for the treatment of a neurological disorder, comprising administering to said patient a neuroprotective amount of an RDP-58 composition.

26. The method according to claim 25, wherein said cell is a neural stem cell.

27. The method according to claim 25, wherein said cell is a fetal cell.

28. The method according to claim 25, wherein said cell is a neuron.

29. The method according to claim 28, wherein said neuron is a dopaminergic neuron or a cholinergic neuron.

30. The method according to claim 25, further comprising contacting said cell with said RDP-58 composition prior to transplanting the cell.


SEQUENCE LISTING (35 sequences)

SEQ ID NO: 1 - protein, 10 aa, Artificial
  Synthetic
RXXXRXXXGY

SEQ ID NO: 2 - protein, 10 aa, Artificial
  Synthetic
XXXXXXXXXY

SEQ ID NO: 3 - protein, 10 aa, Artificial
  Synthetic
RXXXRXXXXY

SEQ ID NO: 4 - protein, 10 aa, Artificial
  Synthetic
RLLLRLLLGY

SEQ ID NO: 5 - protein, 10 aa, Artificial
  Synthetic
RVLLRLLLGY

SEQ ID NO: 6 - protein, 10 aa, Artificial
  Synthetic
RILLRLLLGY

SEQ ID NO: 7 - protein, 10 aa, Artificial
  Synthetic
RLVLRLLLGY

SEQ ID NO: 8 - protein, 10 aa, Artificial
  Synthetic
RLILRLLLGY

SEQ ID NO: 9 - protein, 10 aa, Artificial
  Synthetic
RLLVRLLLGY

SEQ ID NO: 10 - protein, 10 aa, Artificial
  Synthetic
RLLIRLLLGY

SEQ ID NO: 11 - protein, 10 aa, Artificial
  Synthetic
RLLLRVLLGY

SEQ ID NO: 12 - protein, 10 aa, Artificial
  Synthetic
RLLLRILLGY

SEQ ID NO: 13 - protein, 10 aa, Artificial
  Synthetic
RLLLRLVLGY

SEQ ID NO: 14 - protein, 10 aa, Artificial
  Synthetic
RLLLRLILGY

SEQ ID NO: 15 - protein, 10 aa, Artificial
  Synthetic
RLLLRLLVGY

SEQ ID NO: 16 - protein, 10 aa, Artificial
  Synthetic
RLLLRLLIGY

SEQ ID NO: 17 - protein, 10 aa, Artificial
  Synthetic
RWLLRLLLGY

SEQ ID NO: 18 - protein, 10 aa, Artificial
  Synthetic
RLWLRLLLGY

SEQ ID NO: 19 - protein, 10 aa, Artificial
  Synthetic
RLLWRLLLGY

SEQ ID NO: 20 - protein, 10 aa, Artificial
  Synthetic
RLLLRWLLGY

SEQ ID NO: 21 - protein, 10 aa, Artificial
  Synthetic
RLLLRLWLGY

SEQ ID NO: 22 - protein, 10 aa, Artificial
  Synthetic
RLLLRLLWGY

SEQ ID NO: 23 - protein, 10 aa, Artificial
  Synthetic
RYLLRLLLGY

SEQ ID NO: 24 - protein, 10 aa, Artificial
  Synthetic
RLYLRLLLGY

SEQ ID NO: 25 - protein, 10 aa, Artificial
  Synthetic
RLLYRLLLGY

SEQ ID NO: 26 - protein, 10 aa, Artificial
  Synthetic
RLLLRYLLGY

SEQ ID NO: 27 - protein, 10 aa, Artificial
  Synthetic
RLLLRLYLGY

SEQ ID NO: 28 - protein, 10 aa, Artificial
  Synthetic
RLLLRLLYGY

SEQ ID NO: 29 - protein, 5 aa, Artificial
  Synthetic
GSGGS

SEQ ID NO: 30 - protein, 4 aa, Artificial
  Synthetic
GGGS

SEQ ID NO: 31 - protein, 32 aa, Artificial
  Synthetic
XXXXXCXXXCXXXXXXXXXXXXHXXXHXXXXX

SEQ ID NO: 32 - protein, 33 aa, Artificial
  Synthetic
FQCEECXXXXXXXXXXXXXXXXXXXXHIRSHTG

SEQ ID NO: 33 - protein, 30 aa, Artificial
  Synthetic
CXXCXXXXXXXXXXXXXXXXXXXXHXXXXC

SEQ ID NO: 34 - protein, 33 aa, Artificial
  Synthetic
VKCFNCXXXXXXXXXXXXXXXXXXXXHTARNCR

SEQ ID NO: 35 - protein, 34 aa, Artificial
  Synthetic
MNPNCARCGXXXXXXXXXXXXXXXXXXXXHKACF